(19) United States
(12) Patent Application Publication
(10) Pub. No.: US 2025/0242034 A1
(43) Pub. Date: Jul. 31, 2025

(54) PHOSPHORAMIDATE-BASED PSMA-TARGETED SMALL-MOLECULE DRUG CONJUGATES

(71) Applicant: CANCER TARGETED TECHNOLOGY LLC, Woodinville, WA (US)

(72) Inventor: Clifford BERKMAN, Pullman, WA (US)

(21) Appl. No.: 18/833,690

(22) PCT Filed: Feb. 1, 2023
(86) PCT No.: PCT/US2023/061738

§ 371 Date: Jul. 26, 2024

Related U.S. Application Data
(60) Provisional application No. 63/305,770, filed on Feb. 2, 2022.

(51) Int. Cl. A61K 47/54; A61K 49/00; A61K 51/04; A61P 35/00

(52) U.S. Cl. CPC A61K 47/548; A61K 47/545; A61K 49/0002; A61K 51/0497; A61P 35/00; A61K 2123/00

(57) ABSTRACT

The present invention relates to small molecules having high affinity and specificity to prostrate-specific membrane antigen (PSMA) and methods of using them for therapeutic and diagnostic purposes.

### CROSS-REFERENCE TO RELATED APPLICATIONS

This claims priority from U.S. Provisional Application No. 63/305,770, filed Feb. 2, 2022, the disclosure of each of which is incorporated herein by reference in its entirety.

### STATEMENT OF GOVERNMENT INTEREST

This application was supported by Grant No. R21CA223121, awarded by National Institutes of Health. The U.S. government has certain rights in the invention.

### BACKGROUND OF THE DISCLOSURE

### Field of the Invention

The present invention relates to small molecules having high affinity and specificity to prostrate-specific membrane antigen (PSMA) and methods of using them for therapeutic and diagnostic purposes.

### Summary of the Related Art

Prostate cancer (PCa) is the most diagnosed cancer in American men and one of the major causes of cancer-related deaths, second to lung cancers. Generally, patients with localized disease can be treated with radical prostatectomy and/or radiation therapy. Patients with metastatic forms of PCa can be temporarily treated with androgen deprivation strategies, however, most patients with metastatic disease eventually experience disease progression, that can evolve into metastatic castration-resistant prostate cancer (mCRPC), which has a low survival rate. Chemotherapy (e.g., docetaxel, cabazitaxel) is often given following anti-androgen therapy failure, but responses are often marginal, with associated on- and off-target toxicity, especially in elderly men. Better therapeutic approaches are clearly needed for late-stage PCa patients.

Prostate-specific membrane antigen (PSMA) is a type II transmembrane protein that is highly overexpressed by majority of all prostate cancers. PSMA expression is further upregulated in poorly differentiated, metastatic, hormone-refractory carcinomas and in cancer cells from mCRPC patients. Furthermore, PSMA exhibits robust internalization from the cell surface making it an ideal target for imaging and therapy. Indeed, PSMA-targeted radiopharmaceutical therapies have shown promise in clinical trials, with several radioligand therapy and antibody-drug conjugate (ADCs) demonstrating efficacy in preclinical studies.

Problems associated with relapse, off-target toxicity, immunogenicity of antibody-based therapies, as well as the stringent requirements imposed on the facilities that produce and manage radiopharmaceutical production, represent significant challenges for drug development, FDA-approval, and use in the oncology community for agents to treat mCRPC.

Therefore, there remains a need for effective therapies that accurately localize to the prostate cancer lesions without significant off-target toxicity yet are associated with lower manufacturing costs and avoid regulatory challenges associated with radiopharmaceutical manufacturing and handling.

### SUMMARY OF THE DISCLOSURE

Small molecule-drug conjugates (SMDCs) represent an attractive alternative to the more conventional ADC approach for PSMA-targeted chemotherapeutic delivery. Both technologies typically include a PSMA-targeting motif (antibody vs enzyme inhibitor), a spacer, a cleavable linker, and a potent cytotoxic payload. After binding to cell-surface PSMA, these agents are expected to internalize and accumulate in endosomes and lysosomes, which enables efficient release of the cytotoxic payload in the target cells, typically by enzymatic cleavage. Compared to anti-PSMA antibodies, however, small-molecule PSMA inhibitors exhibit similarly accurate localization to the prostate cancer lesions, but their considerably lower molecular weight and simpler molecular characterization is associated with lower manufacturing costs, flexibility in determining the optimal dose regimen, and higher tolerated doses. The toxicity profile of PSMA-SMDCs is expected to be much lower than ADCs, as they have shorter residence time and undergo more rapid and uniform diffusion into the tumor mass compared to normal organs such as kidneys, lacrimal glands, and salivary glands.

Thus, provided herein are therapeutics and diagnostics for prostate cancer that capitalize on the potency and specific affinity of small-molecule inhibitors to PSMA. Accordingly, in one aspect the present disclosure provides compounds of formula (I)

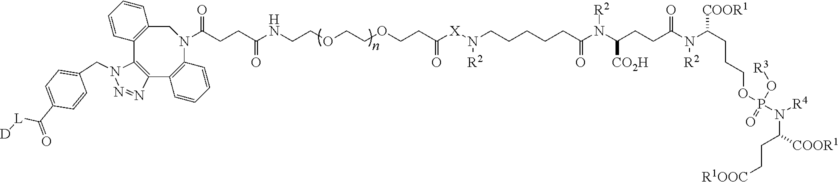

or a pharmaceutically acceptable salt thereof, wherein
D is a therapeutic or diagnostic agent attached to L through —NR²—, —S—, or —O— moiety;
L is an acid-cleavable linker;
X is a bond or

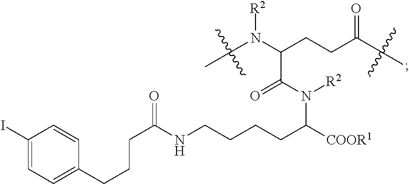

n is in a range of 1 to 6; and
R¹, R², R³, and R⁴ are independently H, -C₁-C₆alkyl, or a protecting group.

The disclosure also provides pharmaceutical compositions comprising the compounds of the disclosure as disclosed herein and a pharmaceutically acceptable excipient, carrier, adjuvant, stabilizer, and/or diluent.

One aspect of the disclosure provides methods of delivering a therapeutic or diagnostic agent to a subject. Such methods include administering a therapeutically effective amount of an effective amount of a compound of the disclosure as disclosed herein or a pharmaceutical composition of the disclosure as disclosed herein to a subject in need of such agent.

One aspect of the disclosure provides methods of treating a patient with cancer (for example, prostate cancer). Such methods include administering to the patient an effective amount of a compound of the disclosure as disclosed herein or a pharmaceutical composition of the disclosure as disclosed herein.

Another aspect of the disclosure provides methods of imaging one or more cancer cells (such as prostate cancer cells) in a patient. Such methods include administering to the patient an effective amount of a compound of the disclosure as disclosed herein or a pharmaceutical composition of the disclosure as disclosed herein.

In another aspect, the present disclosure provides a method of synthesizing the compounds of formula (I). Such methods include contacting a compound of formula (II):

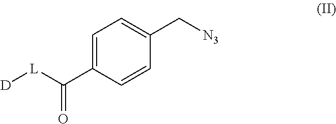

or a pharmaceutically acceptable salt thereof,

with a compound of formula (III):

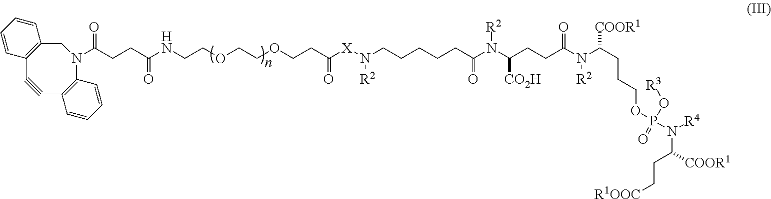

or a pharmaceutically acceptable salt thereof, wherein D, L, n, R¹, R², R³, and
R⁴ are as provided above with respect to formula (I).

Other features and advantages of the present disclosure will become apparent from the following detailed description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiments of the disclosure and, together with the description, serve to explain the principles and operation of the disclosure.

FIG. 1 provides chemical structures of SMDC 1, having Val-Cit-linker, and SMDC 2, having phosphoramidate linker.

FIG. 2 shows therapeutic efficacy of SMDCs 1 and 2. Tumor growth of mice treated with multiple doses of (A) saline (B) MMAE (C) CTT1700, SMDC control equipped with non-cleavable phosphoramidate (D) SMDC 1 (E) SMDC 2 (F) Survival data for mice treated with a multiple dose of controls and SMDC 1 and SMDC 2.

FIG. 3 shows concentrations of parent drug (black circles) and MMAE payload (triangles) after IV dosing of 0.8 mg/kg CTT1700 (left column), SMDC 1 (middle column), or SMDC 2 (right column) to NCr nude mice with PC3-PiP tumors, in plasma (A,B,C) and tumor (D,E,F).

FIG. 4 provides a representative synthesis of SMDC 1 and SMDC 2.

### DETAILED DESCRIPTION

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosed compositions and methods provide improvements in treatment of cancer, particularly prostate cancer, capitalizing on the potency and specific affinity of small-molecule inhibitors of PSMA.

Thus, one aspect of the disclosure provides compounds having the structural formula (I) as described herein.

In certain embodiments, the disclosure provides compounds where X is

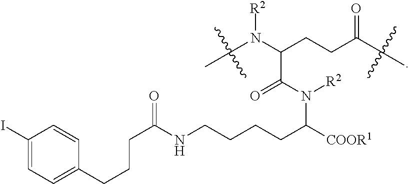

Such compounds can be represented by the formula:

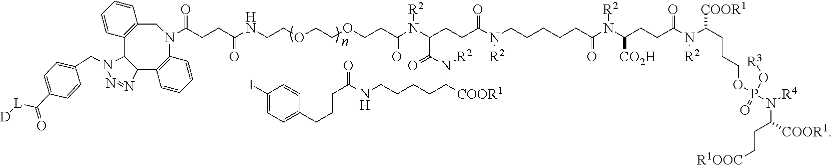

The disclosure, in certain embodiments, provides compounds where X is a bond. Such compounds can be represented by the formula:

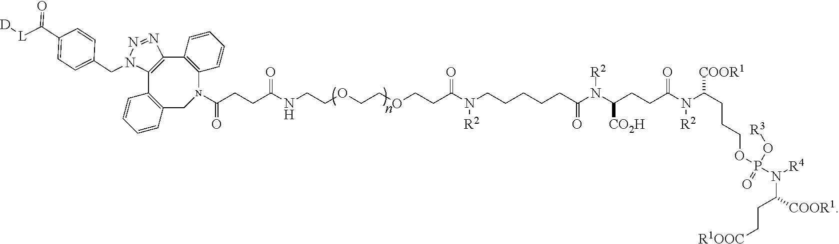

The compounds of the disclosure as described herein include an acid-cleavable linker L (e.g., the linker that decomposes in acidic environment or other desired conditions to release the therapeutic or diagnostic agent). Such linkers when attached to the agent can utilize the lower intracellular pH (for example, pH 5.5) or extracellular pH (for example, pH 6.5) of target cell types, compared to that of blood or normal tissues, to trigger the controlled release of the agent.

In certain embodiments of the disclosure, the linker L comprises a phosphoramidate group. For example, in certain embodiments, L comprises the phosphoramidate of formula:

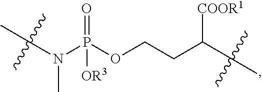

and R¹ and R³ are as provided above with respect to formula (I). In certain embodiments, R¹ is hydrogen, and/or R³ is hydrogen.

In certain embodiments of the disclosure, L is selected from:

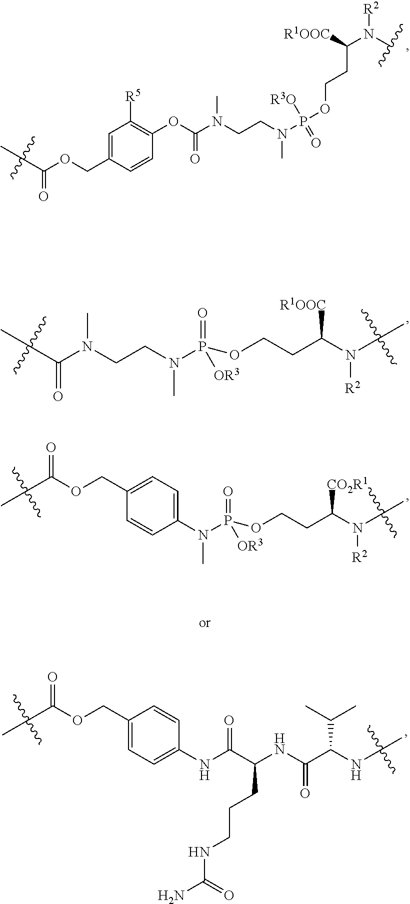

where R⁵ is H, —OH, or —OC₁-C₆alkyl, and R¹, R², and R³ are as provided above with respect to formula (I).

In certain embodiments of the disclosure, L is selected from:

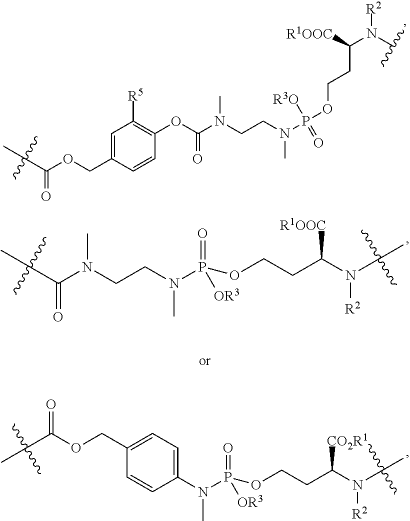

where R⁵ is H, —OH, or —OC₁-C₆ alkyl.

In certain embodiments of the disclosure, L is

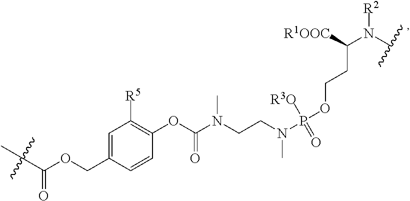

where R⁵ is H, —OH, or —OC₁-C₆alkyl.

In certain embodiments of the disclosure, R⁵ is H, —OH, or —OC₁-C₄ alkyl. In some embodiments, R⁵ is H, —OH, or —OC₁-C₂ alkyl. In some embodiments, R⁵ is H, —OH, or —OCH₃. In some embodiments, R⁵ is H or —OCH₃. In some embodiments, R⁵ is H. In some embodiments, R⁵ is —OCH₃.

In certain embodiments of the disclosure, L is

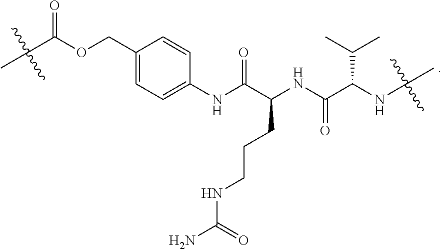 [SMILES: *N[C@H](C(=O)N[C@@H](CCCNC(N)=O)C(=O)Nc1ccc(COC(*)=O)cc1)C(C)C]

The compounds of the disclosure as described herein, in certain example embodiments, are of formula:

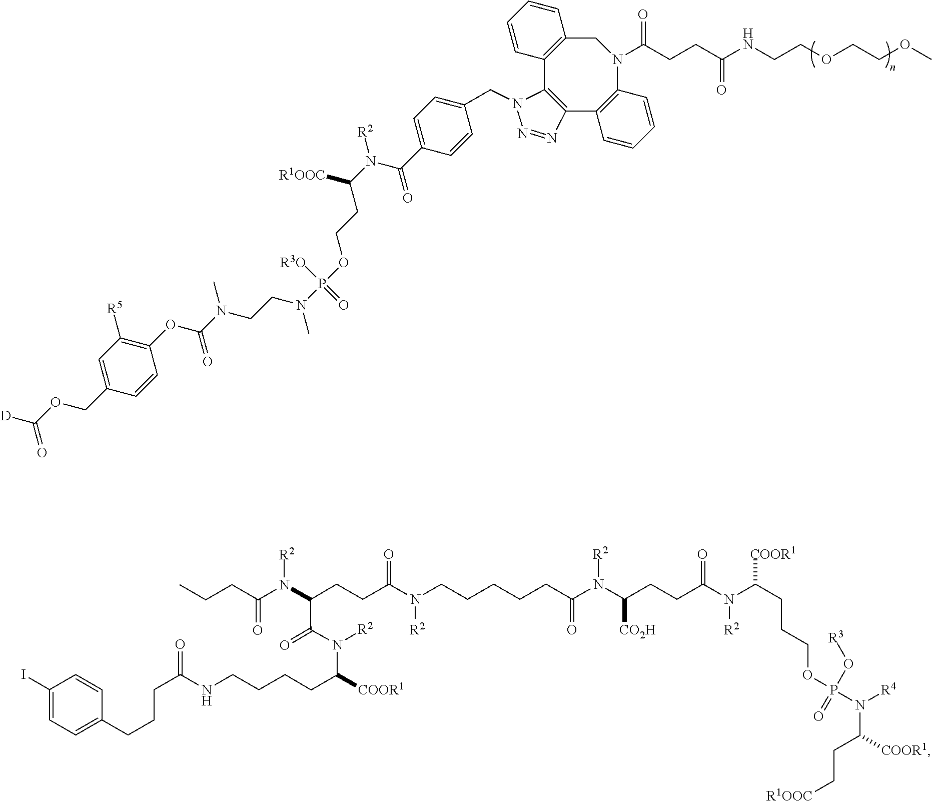

where R⁵ is H, —OH, or —OC₁-C₆ alkyl. In certain embodiments, R⁵ is H, —OH, or —OC₁-C₄ alkyl. In some embodiments, R⁵ is H, —OH, or —OC₁-C₂ alkyl. In some embodiments, R⁵ is H, —OH, or —OCH₃. In some embodiments, R⁵ is H or —OCH₃. In some embodiments, R⁵ is H. In some embodiments, R⁵ is —OCH₃.

In certain embodiment, the compounds are of any of the previous embodiments wherein R¹, R², and R³ are independently selected from one of groups (a)-(o):
(a) hydrogen, C₁-C₆ alkyl or a protecting group.
(b) hydrogen or C₁-C₆ alkyl.
(c) C₁-C₆ alkyl or a protecting group.
(d) C₁-C₆ alkyl.
(e) hydrogen or a protecting group.
(f) hydrogen.
(g) a protecting group.
(h) Any of groups (a)-(d), where C₁-C₆ alkyl is methyl, ethyl, n-propyl, iso-propyl, n-butyl, sec-butyl, tert-butyl, n-pentyl or n-hexyl.
(i) Any of groups (a)-(d), where C₁-C₆ alkyl is methyl, ethyl, n-propyl, iso-propyl, n-butyl, sec-butyl or tert-butyl.
(j) Any of groups (a)-(d), where C₁-C₆ alkyl is methyl, ethyl, n-propyl or tert-butyl.
(k) Any of groups (a)-(d), where C₁-C₆ alkyl is methyl, ethyl or tert-butyl.
(l) Any of groups (a)-(d), where C₁-C₆ alkyl is methyl or ethyl.
(m) Any of groups (a)-(d), where C₁-C₆ alkyl is methyl.
(n) Any of groups (a)-(d), where C₁-C₆ alkyl is ethyl.
(o) Any of groups (a)-(d), where C₁-C₆ alkyl is tert-butyl.

In certain embodiments of the compounds of the disclosure as described herein, R¹, R², and R³ are H.

In certain embodiments, at least one of R¹, R², and R³ is not H, and the remaining R¹, R², and R³ are H.

In certain embodiments, the compounds are of any of the previous embodiments wherein R⁴ is H or methyl. In certain embodiments, R⁴ is H.

In certain embodiments, the disclosure provides the following example compounds represented by the formula:

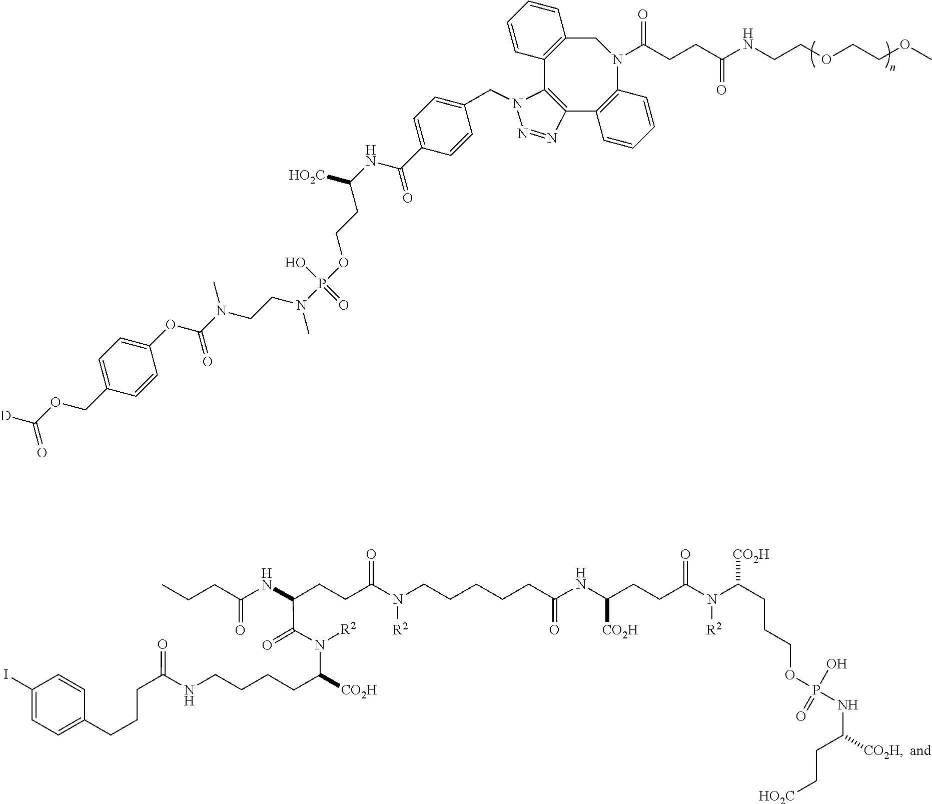

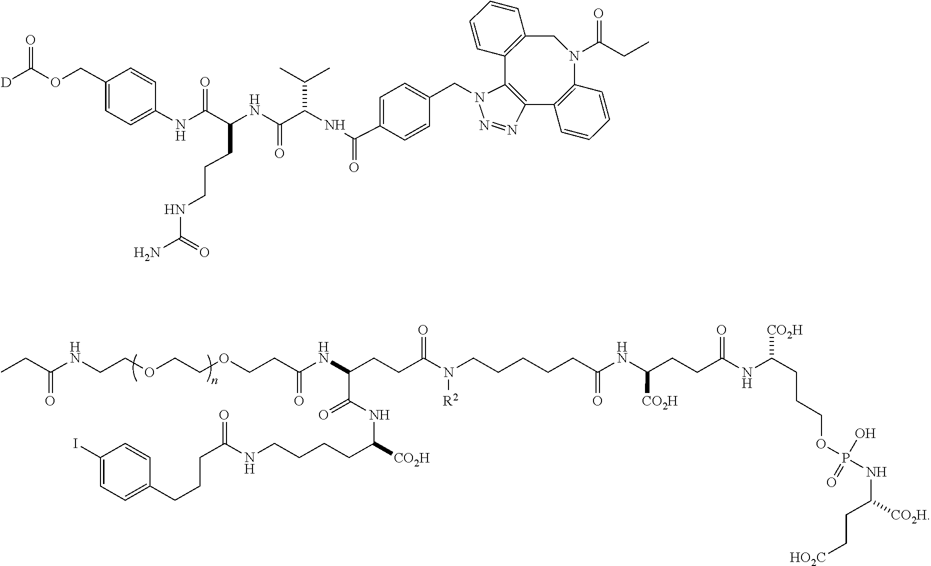

In certain embodiments, the compounds are of any of the previous embodiments wherein n is in a range of 2 to 4. In certain embodiments, n is 3.

As provided above, the compounds of formula (I) as described herein include a therapeutic or diagnostic agent moiety, D. In general, D is attached to the linker L through —NR²—, —S—, or —O—. It is to be understood that the agents may actually be derivatives, with modifications at the linking site. For example, D can be modified to comprise —NR²—, —S—, or —O— for attaching to L.

A therapeutic agent is a molecule that is useful in the treatment of a disease. Examples of therapeutic agents include chemotherapeutic agents, antibodies, antibody fragments, toxins, enzymes, nucleases such as a ribonuclease (RNase) or DNase I, hormones, cytokines, chemokines, angiogenesis inhibitors, antisense oligonucleotides, small interfering RNA (siRNA), chelators, boron compounds, photoactive agents, small molecules, antibiotics, and radioisotopes.

A chemotherapeutic agent includes, for example, an anticancer agent, an antineoplastic agent, and a cytotoxic agent. Examples of anti-cancer chemotherapeutic agents include, but are not limited to, 5-fluorouracil, bleomycin, busulfan, camptothecins, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, methotrexate, mitomycin, navelbine, nitrosurea, plicamycin, procarbazine, raloxifene, tamoxifen, TAXOL, temazolomide (an aqueous form of DTIC), transplatinum, vinblastine and methotrexate, vincristine, or any analog or derivative variant of the foregoing. Chemotherapeutic agents of use against infectious organisms include, but are not limited to, acyclovir, albendazole, amantadine, amikacin, amoxicillin, amphotericin B, ampicillin, aztreonam, azithromycin, bacitracin, BACTRIM, BATRAFEN, bifonazole, carbenicillin, caspofungin, cefaclor, cefazolin, cephalosporins, cefepime, ceftriaxone, cefotaxime, chloramphenicol, cidofovir, Cipro®, clarithromycin, clavulanic acid, clotrimazole, cloxacillin, doxycycline, econazole, erythrocycline, erythromycin, FLAGYL®, fluconazole, flucytosine, FOSCARNET®, furazolidone, ganciclovir, gentamycin, imipenem, isoniazid, itraconazole, kanamycin, ketoconazole, lincomycin, linezolid, meropenem, miconazole, minocycline, naftifine, nalidixic acid, neomycin, netilmicin, nitrofurantoin, nystatin, oseltamivir, oxacillin, paromomycin, penicillin, pentamidine, piperacillin-tazobactam, rifabutin, rifampin, rimantadine, streptomycin, sulfamethoxazole, sulfasalazine, tetracycline, tioconazole, tobramycin, tolciclate, tolnaftate, trimethoprim sulfamethoxazole, valacyclovir, vancomycin, zanamir, and zithromycin.

Hormones can be used as a therapeutic agent themselves or in combination with other chemotherapeutic agents. Progestins, such as hydroxyprogesterone caproate, medroxyprogesterone acetate, and megestrol acetate, have been used in cancers of the endometrium and breast. Estrogens such as diethylstilbestrol and ethinyl estradiol have been used in cancers such as prostate cancer. Antiestrogens such as tamoxifen have been used in cancers such as breast cancer. Androgens such as testosterone propionate and fluoxymesterone have also been used in treating breast cancer. Corticosteroid hormones such as prednisone and dexamethasone can improve the effective of other chemotherapeutic agents. Cytokines that are used as therapeutic agents include, but are not limited to, lymphokines, monokines, growth factors, and polypeptide hormones. Examples of cytokines include but are not limited to human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, tumor necrosis factor-α, tumor necrosis factor-β, mullerian-inhibiting substance, mouse gonadotropin-associated peptide, inhibin, activin, vascular endothelial growth factor, integrin, thrombopoietin (TPO), NGF-β, platelet-growth factor, TGF-α, TGF-β, insulin-like growth factor-I, insulin-like growth factor-II, erythropoietin (EPO), osteoinductive factor, interferon-α, interferon-β, interferon-γ, macrophage-CSF (M-CSF), granulocyte-macrophage-CSF (GM-CSF), granulocyte-CSF (G-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, LIF, kit-ligand, FLT-3, angiostatin, thrombospondin, endostatin, and lymphotoxin. Examples of angiogenesis inhibitors that are used as therapeutic agents include, but are not limited to, angiostatin, baculostatin, canstatin, maspin, anti-VEGF antibodies, anti-PIGF peptides and antibodies, anti-vascular growth factor antibodies, anti-Flk-1 antibodies, anti-Flt-1 antibodies and peptides, laminin peptides, fibronectin peptides, plasminogen activator inhibitors, tissue metalloproteinase inhibitors, interferons, interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4, and minocycline. Examples of small molecules for use as therapeutic agents include, but are not limited to, abrin, amantadine, amoxicillin, amphotericin B, ampicillin, aplidin, azaribine, anastrozole, azacytidine, aztreonam, azithromycin, bacitracin, trimethoprim/sulfamethoxazole, Batrafen, bifonazole, bleomycin, bortezomib, bryostatin-1, busulfan, calicheamycin, camptothecin, 10-hydroxycamptothecin, carbenicillin, caspofungin, carmustine, cefaclor, cefazolin, cephalosporins, cefepime, ceftriaxone, cefotaxime, celecoxib, chlorambucil, chloramphenicol, ciprofloxacin, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, diphtheria toxin, DNase I, doxorubicin, 2-pyrrolinodoxorubicine (2P-DOX), doxycycline, cyano-morpholino doxorubicin, doxorubicin glucuronide, duocarmycin (DUBA), epirubicin glucuronide, ethinyl estradiol, 7-ethyl-10-hydroxycamptothecin (SN-38), estramustine, estrogen receptor binding agents, etoposide, etoposide glucuronide, etoposide phosphate, erythrocycline, erythromycin, flagyl, farnesyl-protein transferase inhibitors, floxuridine (FUdR), 3′,5′-O-dioleoyl-FudR (FUdR-dO), fludarabine, flutamide, fluorouracil, fluoxymesterone, ganciclovir, gentamycin, gelonin, gemcitabine, hydroxyprogesterone caproate, hydroxyurea, idarubicin, ifosfamide, imiquimod, isoniazid, itraconazole, kanamycin, ketoconazole, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, minocycline, naftifine, nalidixic acid, neomycin, navelbine, nitrosurea, nystatin, ranpirnase, oxacillin, paromomycin, penicillin, pentamidine, piperacillin-tazobactam, phenyl butyrate, prednisone, procarbazine, paclitaxel, pentostatin, pokeweed antiviral protein, PSI-341, seco-duocarmycin (seco-DUBA), semustine, rifabutin, rifampin, rimantadine, streptomycin, sulfamethoxazole, sulfasalazine, streptozocin, tamoxifen, taxanes, taxol, testosterone propionate, tetracycline, thalidomide, thioguanine, thiotepa, teniposide, topotecan, transplatinum, trimethoprim sulfamethoxazole, uracil mustard, valacyclovir, vancomycin, vinblastine, vinorelbine, vincristine, zanamir, zithromycin, and (R)-5-chloro-N²-[4-(4-methylpiperazin-1-yl)phenyl]-N⁴-[(tetrahydrofuran-2-yl)methyl]pyrimidine-2,4-diamine ((R)-9b).

In embodiments, D disclosed herein include therapeutic agents for the treatment of cancer and non-cancer therapeutic agents. These therapeutic agents include organic small molecules: including all hydroxyl and amine-containing therapeutic agents for the treatment of cancer, for example, molecules that inhibit the replication of DNA (e.g., doxorubicin, epirubicin, calecheamicin, camptothecin), molecules that stabilize or disrupt microtubules (e.g., paclitaxel, docetaxel, epothilone), molecules that affect the Na⁺/K⁺ pump (e.g., strophanthidin), molecules that affect the function of the Golgi apparatus (e.g., norrisolide and active derivatives of norrisolide). These therapeutic agents also include inorganic small molecules, such as all hydroxyl and amine containing therapeutic agents for the treatment of cancer, for example, cisplatin or oxoplatin. Examples of linked anti-tumor agents include, for example, CO-doxorubicin, and CO-strophanthidin.

In other embodiments, D disclosed herein include but are not limited to proteins: including proteins of human and non-human origin, for example, antibodies (e.g. trastuzumab), hormones (e.g. leutinizing hormone, follicle stimulating hormone), cytokines (e.g. IL-6), growth factors (e.g. G-CSF), bacterial or plant toxins (e.g., Pseudomanas toxin, gelonin, ricin, abrin) and tumor-targeting soluble proteins of any type; peptides including engineered and natural peptides that are toxic to tumor cells, that alter the architecture or function of such cells, or target other molecules to tumor cells or cells in the tumor that serve to support tumor cells, for example, lysins, TAT-related proteins that enhance cell penetration; nucleic acids such as RNA, for example, anti-sense RNA, silencing RNA, toxin aptamers, DNA such as naturally-occurring and synthetic oligonucleotides and higher molecular weight structures, for example, plasmid and viral vectors that express RNAs or proteins that are toxic to tumor cells; particles such as polymer-derived, protein-derived, metal-derived and inorganic-based particles of any size, for example, nanoparticles loaded with therapeutic agents, detectable labels or imaging agents such as fluorescent dyes or radionuclides; small molecules such as both inorganic and organic small molecules that target cell surface receptors or otherwise bind to the surface or other accessible intracellular or extracellular components of tumor cells.

Therapeutic agents also include drugs that are active in the CNS, for example, L-Dopa, Ritalin, Cymbalta, Namenda, and Gleevec.

In other embodiments, D is an anticancer agent, an antineoplastic agent, or a cytoxic molecule. In one embodiment, D is selected from the group consisting of an amine group containing antineoplastic agent say, for example, monomethylauristatin E (MMAE), monomethylauristatin F (MMAF), and doxorubicin.

In one embodiment, D is selected from MMAE, MMAF, doxorubicin, cabazitaxel, docetaxel, paclitaxel, gemcitabine, imiquimod, SN-38, DUBA, seco-DUBA, (R)-9b, and gemcitabine.

A diagnostic agent is a molecule that may be used in imaging studies such as magnetic resonance imaging (MRI), magnetic resonance tomography (MRT), positron emission tomography (PET), computer tomography (CT), single-photon emission computed tomography (SPECT) and optical imaging, such as x-ray. Diagnostic agents are detectable or traceable labels. Examples of diagnostic agents used in these studies include, but are not limited to, radioisotopes, dyes (including those using a biotin-streptavidin complex), enzymes, contrast agents, fluorescent compounds or molecules such as a fluorescent dye, paramagnetic ions (for MRI), and small molecules including both inorganic and organic small molecules that target cell surface receptors or otherwise bind to the surface or other accessible intracellular or extracellular components of tumor cells.

In certain embodiments, D is a radioisotope, an imaging agent, a fluorescent dye, a near-IR dye, an enzyme, a chemiluminescent agent, a bioluminescent agent, a paramagnetic ion, an ultrasound label, or a radioacoustic label.

In one aspect, the present disclosure provides pharmaceutical compositions comprising a compound of the disclosure as described herein and a pharmaceutically acceptable excipient, carrier, adjuvant, stabilizer, and/or diluent. Pharmaceutically acceptable excipient, carrier, adjuvant, stabilizer, diluent, etc. to be included are determined by the composition being administered and by the method of administering the composition. There are a wide variety of suitable formulations of pharmaceutical composition including optional pharmaceutically acceptable carriers, excipients, stabilizers, etc. Acceptable carriers or diluents for therapeutic use are well known in the pharmaceutical art, and are described, for example, in Remington's Pharmaceutical Sciences, 18th Ed., Mack Publishing Co., Easton, Pa. (1990).

Pharmaceutical compositions suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically or intrathecally.

The pharmaceutical compositions disclosed herein may be manufactured by means of conventional mixing, dissolving, granulating, dragee-making, levigating, emulsifying, encapsulating, entrapping or tableting processes. The pharmaceutical compositions can be prepared as liquid solutions or suspensions, solid forms suitable for solution or suspension in liquid prior to use, or as emulsions.

In certain aspects, the disclosure also provides methods of using the disclosed compounds for therapeutic and diagnostic purposes. For example, the disclosure provides methods of treating or ameliorating a disease or condition that can include administering an effective amount of one or more of the compounds as described herein or one or more of the pharmaceutical compositions as described herein to a subject in need thereof.

In embodiments, the compounds of the disclosure deliver an anticancer drug to a selected tissue. Cancer can be lung cancer, breast cancer, colon cancer, ovarian cancer, prostate cancer, and melanoma.

Thus, in certain embodiments, the disclosure provides methods of treating a patient with prostate cancer by administering an effective amount of compound as described herein or the pharmaceutical composition as described herein to the patient. The amount of the compound and regiment can be routinely determined using art-recognized techniques.

In certain embodiments, the disclosure provides methods for imaging one or more cancer cells (such as prostate cancer cell) in a patient by administering to the patient an effective amount of compound as described herein or the pharmaceutical composition as described herein. The method may further include imaging the compound in vivo. The imaging can be performed with any imaging techniques known in the art.

### Definitions

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” PSMA with a compound includes the administration of a compound described herein to a subject or patient, such as a human, as well as, for example, introducing a compound into a sample containing a cellular or purified preparation containing PSMA.

As used herein, the term “subject” or “patient,” used interchangeably, refers to any animal, including mammals, preferably mice, rats, other rodents, rabbits, dogs, cats, swine, cattle, sheep, horses, or primates, and most preferably humans.

As used herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. In certain embodiments, the pharmaceutically acceptable salt is a sodium salt. Those skilled in the art will recognize a wide variety of non-toxic pharmaceutically acceptable addition salts.

The term “alkyl” as used herein, means a straight or branched chain hydrocarbon containing from 1 to 10 carbon atoms, unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, —CH₂CH(CH₂CH₃)CH₂—.

A “protecting group” as used herein include, but are not limited to, optionally substituted benzyl, t-butyl ester, allyl ester, alkyl esters (e.g., methyl, ethyl), fluorenylmethoxycarbonyl groups (Fmoc), and amino, carboxylic acid and phosphorus acid protecting groups described in Greene's Protective Groups in Organic Synthesis, 4th Edition (which is incorporated by reference). In some embodiments, R¹ is a carboxylic acid protecting group (e.g., a methyl or t-butyl ester). In some embodiments, R² is a nitrogen protecting group (e.g., Boc, or benzyl). Optionally benzyl groups include, but are not limited to, unsubstituted benzyl, triphenylmethyl (trityl), diphenylmethyl, o-nitrobenzyl, 2,4,6-trimethylbenzyl, p-bromobenzyl, p-nitrobenzyl, p-methoxybenzyl (PMB), 2,6-dimethoxybenzyl, 4-(methylsulfinyl)benzyl, 4-sulfobenzyl, 4-azidomethoxybenzyl, and piperonyl, and benzyl protecting groups for carboxylic and phosphorus acids disclosed in Greene's Protective Groups in Organic Synthesis (the relevant parts of which are incorporated by reference).

### EXAMPLES

The compounds disclosed herein can be made using procedures familiar to the person of ordinary skill in the art and as described herein. For example, compounds of structural formula (I) can be prepared according to general procedures (below), and/or analogous synthetic procedures. One of skill in the art can adapt the reaction sequences of Examples 1 and 2, and general procedures to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

The compounds and methods of the disclosure are illustrated further by the following examples, which are not to be construed as limiting the disclosure in scope or spirit to the specific procedures and compounds described in them.

### Development of SMDC 1 and SMDC 2

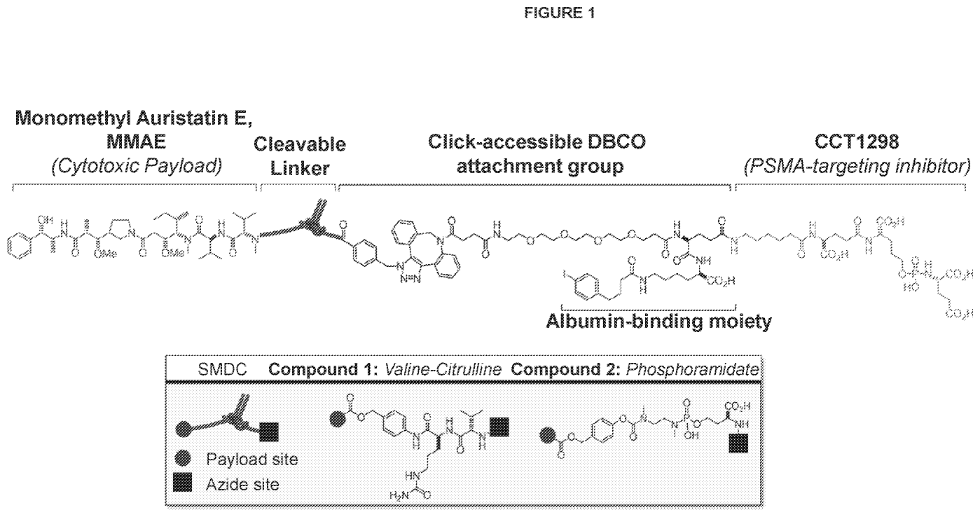

Two PSMA-targeted small molecule-drug conjugates, SMDC 1 and SMDC 2, FIG. 1, were prepared. In compound 1, a valine-citrulline (Val-Cit) dipeptide linker was utilized. This linker is cleavable by lysosome-abundant cathepsin B proteases to release the cytotoxic payload. In compound 2, we used an acid-labile phosphoramidate linker. This linker exhibits stability in the neutral pH of the plasma, with rapid release of the payload in the acidic conditions of endosomal and lysosomal compartments. Monomethyl auristatin E (MMAE), an anti-tubulin agent that kills cells at sub-nanomolar concentrations by inhibiting the assembly of microtubules followed by subsequent induction of mitotic arrest, was used as the cytotoxic payload for both SMDCs. In both SMDCs, a phosphoramidate-based inhibitor, CTT1298, was selected as the PSMA-targeting molecule. This is due to its documented high-affinity, irreversible binding to PSMA (IC₅₀=19 nM) and high selectivity for PSMA-expressing cancer cells. Once bound to extracellular PSMA, CTT1298 derivatized with radiolabeled pendant groups has been shown to undergo rapid and extensive internalization in PSMA-expressing tumor cells. Moreover, conjugation of chemical moieties to CTT1298 does not diminish the binding affinity of this ligand to PSMA.

One of the major challenges for small-molecule PSMA inhibitors with respect to delivering therapeutic payloads is their rapid renal clearance. This is due to the small molecular weight and highly charged nature (poly-carboxylate) of the inhibitor structures, but this charge is also necessary for their high affinity to PSMA. To overcome this pharmacokinetic challenge, the two SMDCs comprise an albumin-binding motif known to reduce receptor-mediated kidney uptake and increase tumor uptake of drug-conjugates.

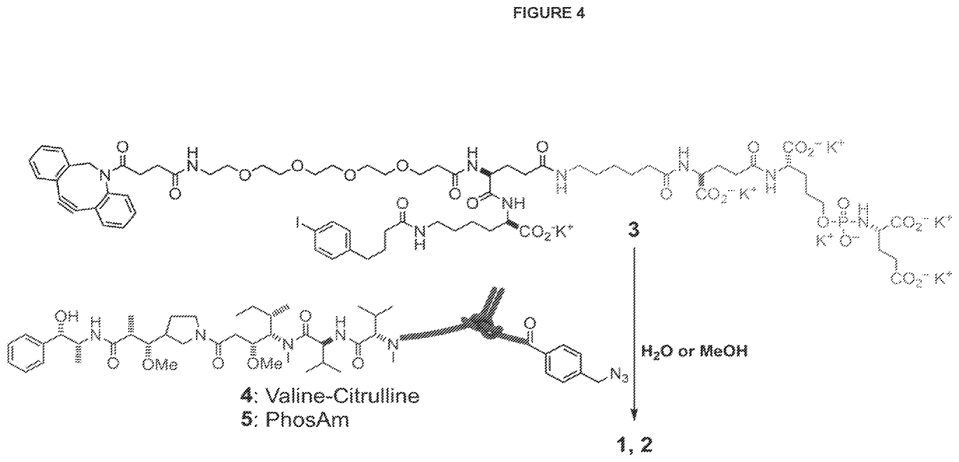

The synthesis of SMDC 1 and SMDC 2 were achieved through copper-free click reaction (see FIG. 4) as provided in more detail below. One key precursor was compound 3, which contains the strained dibenzocyclooctyne for click chemistry, CTT1298 as the targeting molecule, and the albumin-binding moiety into its molecular structure. Azides 4 and 5 incorporated the Val-Cit and phosphoramidate linkers, respectively, in addition to the cytotoxic payload. These click-chemistry reactions were monitored by HPLC and found to be complete in less than 30 minutes. SMDC 1 and SMDC 2 were purified by preparative C18-HPLC and further desalted to give isolated yields of 30% and 49%, respectively, and were fully characterized by HRMS, HPLC and ³¹P NMR.

### Example 1. Preparation and Characterization of SMDC 1

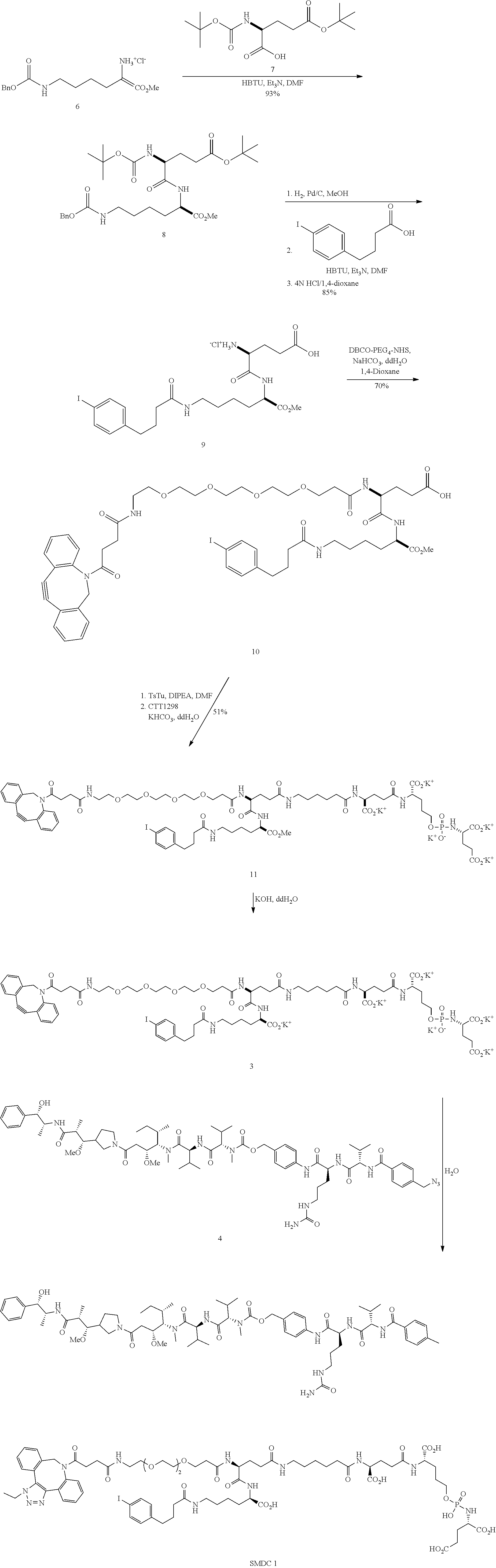

All anhydrous solvents used in reactions were obtained from commercial sources or freshly distilled over calcium hydride. ¹H, ¹³C, and ³¹P NMR spectra were recorded on a Varian 400, Brucker Avance Neo 500, or Varian 600 MHz spectrometer. ¹H NMR chemical shifts are relative to CDCl₃ (δ=7.26 ppm), CD₃OD (δ=3.31 ppm) or D₂O (δ=4.79 ppm). ¹³C NMR chemical shifts were relative to CDCl₃ (δ=77.23 ppm) or CD₃OD (δ=49.15 ppm). High-resolution mass spectrometry (HRMS) spectra were obtained on an Applied Biosystems 4800 MALDI-TOF/TOF mass spectrometer (Applied Biosystems, Foster City, CA).

### Methyl N2-((S)-5-(tert-butoxy)-2-((tert-butoxycarbonyl)amino)-5-oxopentanoyl)-N6-(4-(4-iodophenyl) butanoyl)-D-lysinate (8)

A solution of acid, 7 (2 g, 6.59 mmol) and HBTU (2.5 g, 6.59 mmol) was stirred in anhydrous DMF (11 mL) under Ar at room temperature for 30 min. A solution of 6 (2.6 g, 7.91 mmol) and anhydrous Et₃N (2.5 mL, 16.5 mmol) in DMF (11 mL) was added to the reaction solution, and the reaction was stirred for 3 h under Ar. The reaction was diluted with EtOAc (150 mL) and washed sequentially with 1 N HCl (75 mL, 3×), sat. NaHCO₃ (aq) (75 mL, 2×) and Brine (75 mL, 1×). Organic layer was dried on Na₂SO₄, volatiles were removed under reduced pressure, resulting residue was dried under high vacuum and carried on to the next step without further purification. Isolated yield was 93%. ¹H NMR (400 MHz, DMSO-d₆) δ 8.14 (d, J=7.7 Hz, 1H), 7.40-7.28 (m, 5H), 7.23 (t, J=5.7 Hz, 1H), 6.82 (d, J=8.3 Hz, 1H), 4.99 (s, 2H), 4.24-4.14 (m, 1H), 4.03-3.92 (m, 1H), 3.61 (s, 3H), 2.95 (q, J=6.5 Hz, 2H), 2.20 (q, J=6.6, 5.1 Hz, 2H), 1.81 (dq, J=13.4, 6.8 Hz, 1H), 1.67 (m, 3H), 1.38(2s, 18H), 1.30-1.20 (m, 2H). ¹³C NMR (126 MHz, CDCl₃) δ 172.9, 172.6, 171.7, 156.6, 155.8, 136.7, 128.6, 128.5, 128.2, 128.1, 81.1, 80.2, 66.7, 54.3, 52.5, 51.9, 40.6, 39.4, 31.9, 31.8, 28.6, 28.3, 28.2, 27.3, 22.3, 21.2. HRMS (MALDI): m/z calculated for C₂₉H₄₅KN₃O₉⁺ [M+K]⁺: 618.27930, found 618.27661.

### (S)-4-carboxy-1-(((R)-6-(4-(4-iodophenyl)butanamido)-1-methoxy-1-oxohexan-2-yl)amino)-1-oxobutan-2-aminium chloride (9)

An evacuated flask containing 8 (3.4 g, 5.86 mmol) and Pd/C (0.34 g, 10% mass eq) in MeOH (60 mL) was charged with H2 under balloon pressure. The reaction was stirred continuously until complete by TLC. Pd/C was filtered off using a celite pad, filtrate was concentrated under reduced pressure, the leftover residue was dried under high vacuum and carried on to the next step without further purification. A solution of 4-(4-iodophenyl)butanoic acid (1.7 g, 5.86 mmol) and HBTU (2.23 g, 5.86 mmol) was stirred in anhydrous DMF (10 mL) under Ar at room temperature for 30 min. A solution of CBz-deprotected amine (2.74 g, 6.15 mmol) and anhydrous Et₃N (1.53 mL, 8.79 mmol) in DMF (10 mL) was added to the reaction solution, and the reaction was stirred for 6 h under Ar. The reaction was diluted with EtOAc (150 mL) and washed sequentially with 1N HCl (75 mL, 3×), sat. NaHCO₃ (aq) (75 mL, 2×) and Brine (75 mL, 1×). Organic layer was dried on Na₂SO₄, volatiles were removed under reduced pressure, resulting residue was dried under high vacuum and carried on to the next step without further purification. Isolated yield was 85%. Pre-9 ¹H NMR (400 MHz, DMSO-d₆) δ 8.13 (d, J=7.7 Hz, 1H), 7.74 (t, J=5.6 Hz, 1H), 7.62 (d, J=8.2 Hz, 2H), 7.00 (d, J=8.2 Hz, 2H), 6.82 (d, J=8.3 Hz, 1H), 4.19 (td, J=8.5, 5.2 Hz, 1H), 3.99 (dt, J=22.0, 7.6 Hz, 1H), 3.60 (s, 3H), 2.98 (hept, J=6.3 Hz, 2H), 2.19 (q, J=6.8, 6.1 Hz, 2H), 2.03 (t, J=7.4 Hz, 2H), 1.69 (m, 5H), 1.38 (2s, 18H), 1.24 (q, J=7.0 Hz, 2H). Pre-9 ¹³C NMR (126 MHz, CDCl₃) δ 173.33, 173.03, 172.57, 171.68, 155.86, 141.32, 137.46, 130.73, 91.06, 81.21, 80.33, 54.72, 52.59, 51.75, 39.13, 35.60, 34.82, 31.97, 31.83, 28.55, 28.37, 28.31, 28.16, 27.07, 22.28. Pre-9 HRMS (MALDI): m/z calculated for C₃₁H₄₈IN₃NaO₈⁺ [M+Na]⁺: 740.23783, found 740.24152. In a dry evacuated flask, a solution of Boc-protected amine (0.099 g, 0.137 mmol) was stirred continuous in 4N HCl-1,4-dioxane (1 mL) for 1 h at room temperature. Volatiles were removed via rotary evaporation; the resulting residue was dried under high vacuum to yield an off-white solid in quantitative yield. ¹H NMR (400 MHz, DMSO-d₆) δ 8.95 (d, J=7.5 Hz, 1H), 8.23 (d, J=5.3 Hz, 3H), 7.79 (t, J=5.6 Hz, 1H), 7.64-7.56 (m, 2H), 7.02-6.95 (m, 2H), 4.23 (q, J=7.7 Hz, 1H), 3.85 (d, J=5.8 Hz, 1H), 3.61 (s, 3H), 3.02-2.93 (m, 2H), 2.30 (h, J=10.2, 9.7 Hz, 2H), 1.99 (dt, J=19.2, 7.0 Hz, 4H), 1.69 (dp, J=28.2, 7.5 Hz, 4H), 1.34 (q, J=7.0 Hz, 2H), 1.25 (d, J=8.0 Hz, 2H). ¹³C NMR (126 MHz, DMSO-d₆) δ 173.3, 172.1, 171.7, 168.4, 141.7, 137.0, 130.9, 91.4, 66.4, 52.3, 52.0, 51.5, 38.0, 34.7, 34.1, 30.4, 29.2, 28.6, 26.9, 26.5, 22.7. HRMS (MALDI): m/z calculated for C₂₂H₃₄ClIN₃O₆⁺[M+H]⁺: 598.14160, found 598.14044; m/z calculated for C₂₂H₃₃ClIN₃NaO₆⁺ [M+Na]⁺: 620.12361, found 620.12140.

### Compound 10.

To a stirring of solution of 9 (0.184 g. 0.308 mmol) and NaHCO₃ (0.0801 g, 0.800 mmol) in ddH₂O (2 mL) was added a solution of DBCO-PEG₄-NHS (0.1 g, 0.154 mmol) in 1,4-dioxane (2 mL) dropwise. The reaction was stirred at ambient temperature for 3 h. ddH2O (10 mL) was added to the reaction solution and the pH was slowly adjusted to 2 via 1N HCl. More ddH₂O (10 mL) was added, and the organics were extracted into EtOAc (20 mL, 3×). Organic layer was combined and dried under Na₂SO₄, followed by removal of solvent under reduced pressure and the resulting residue was purified via silica-gel flash chromatography (14% MeOH in CH₂Cl₂→20% MeOH in CH₂Cl₂) to yield a yellow solid in 70% yield. TLC: Rf=0.194 (15% MeOH in CH₂Cl₂, visualization by UV). ¹H NMR (600 MHz, CD₃OD) δ 7.62 (dd, J=7.6, 1.4 Hz, 1H), 7.58-7.52 (m, 2H), 7.45 (m, 1H), 7.43-7.38 (m, 2H), 7.31 (m, 2H), 7.22 (dd, J=7.5, 1.5 Hz, 1H), 6.97-6.92 (m, 2H), 5.09 (d, J=14.0 Hz, 1H), 4.40 (m, 1H), 4.35 (m, 1H), 3.70-3.61 (m, 6H), 3.61-3.46 (m, 13H), 3.25-3.19 (m, 1H), 3.17-3.04 (m, 3H), 2.56-2.38 (m, 5H), 2.32 (td, J=7.3, 3.6 Hz, 2H), 2.30-2.25 (m, 2H), 2.14 (t, J=7.5 Hz, 2H), 2.02 (ddt, J=23.2, 16.0, 7.0 Hz, 2H), 1.92-1.76 (m, 4H), 1.71-1.62 (m, 1H), 1.44 (dp, J=19.9, 6.6 Hz, 2H), 1.32 (m, 2H). ¹³C NMR (151 MHz, CD₃OD) δ 175.6, 174.1, 173.9, 173.8, 173.1, 152.5, 149.5, 142.8, 138.5, 133.5, 131.8, 130.5, 130.0, 129.7, 129.2, 128.9, 128.1, 126.5, 124.3, 123.4, 115.6, 108.7, 91.5, 71.1, 70.9, 68.3, 68.1, 56.56, 54.1, 53.6, 52.8, 49.9, 39.9, 37.2, 37.1, 36.6, 36.3, 35.7, 35.4, 32.1, 32.0, 29.8, 29.7, 28.9, 28.5, 24.2. HRMS (MALDI): m/z calculated for C₅₂H₆₆IN₅NaO₁₃⁺[M+Na]⁺: 1118.35940, found 1118.36194; m/z calculated for C₅₂H₆₆IKN₅O₁₃⁺[M+K]⁺: 1134.33334, found 1134.33423.

### Compound 11.

To a stirring of solution of 42 (0.35 g, 0.319 mmol) and DIPEA (0.068 mL, 0.383 mmol) in anhydrous DMF (6 mL) was added TsTu (0.096 g, 0.319 mmol) in one portion under Argon. The reaction was stirred at ambient temperature for 3 h. A solution of CTT1298 (0.322 g, 0.415 mmol) and KHCO₃ (0.064 g, 0.639 mmol) in ddH2O (4 mL) was added to the reaction solution and the reaction was stirred continuously overnight. Volatiles were removed under reduced pressure resulting residue was purified via reverse-phase flash chromatography (100% H₂O→100% MeOH) to yield a yellow solid in 51% yield. TLC: Rf=0.37 (70% MeOH in H₂O, visualization by UV). ¹H NMR (600 MHz, D₂O) δ 7.69 (d, J=7.6 Hz, 1H), 7.63 (d, J=7.9 Hz, 2H), 7.55-7.41 (m, 6H), 7.37 (d, J=7.6 Hz, 1H), 6.99 (d, J=8.0 Hz, 2H), 5.09 (d, J=14.3 Hz, 1H), 4.37 (dd, J=9.4, 4.9 Hz, 1H), 4.33-4.27 (m, 1H), 4.15 (dd, J=8.7, 4.7 Hz, 1H), 4.10 (dd, J=8.3, 4.9 Hz, 1H), 3.80-3.55 (m, 26H), 3.54-3.47 (m, 1H), 3.20 (dt, J=12.3, 5.7 Hz, 1H), 3.10 (dq, J=28.9, 7.0 Hz, 6H), 2.82 (d, J=1.0 Hz, 1H), 2.56 (m, 4H), 2.50-2.16 (m, 17H), 2.16-2.02 (m, 3H), 1.87 (m, 1OH), 1.72 (m, 3H), 1.65-1.53 (m, 5H), 1.48 (m, 5H), 1.38-1.24 (m, 5H). ³¹P NMR (234 MHz, D₂O) δ 7.32. ¹³C NMR (126 MHz, D₂O) δ 183.9, 182.5, 182.4, 180.2, 179.3, 177.2, 175.9, 175.7, 175.2, 174.7, 174.6, 174.3, 174.0, 173.3, 151.6, 148.7, 142.5, 138.2, 133.2, 131.7, 130.2, 129.7, 129.5, 129.0, 127.9, 126.5, 123.6, 122.6, 115.7, 109.1, 92.3, 70.7, 70.6, 70.6, 70.6, 70.5, 67.8, 67.6, 65.4, 65.3, 57.6, 56.4, 55.8, 54.3, 53.7, 53.5, 40.3, 39.9, 39.7, 36.9, 36.8, 36.6, 36.5, 36.4, 35.3, 34.9, 34.8, 33.4, 33.1, 33.0, 32.9, 31.1, 29.2, 29.1, 29.0, 28.3, 28.1, 27.9, 27.8, 26.8, 25.9, 25.8, 23.6. HRMS (MALDI): m/z calculated for C₇₃H₁₀₁IKN₉O₂₅P⁺ [M+K]⁺: 1700.53225, found 1700.53699.

### Compound 3.

To a stirring of solution of 11 (0.29 g, 0.157 mmol) in ddH₂O (5 mL) was added 1 N KOH dropwise until the pH=12.5. Reaction was stirred until completion by TLC. Reaction solution lyophilized to give an off-white solid and carried on to the next step without further purification. Isolated yield was quantitative. TLC: Rf=0.42 (70% MeOH in H₂O, visualization by UV). ¹H NMR (600 MHz, D₂O) δ 7.25 (d, J=8.1 Hz, 2H), 7.23-7.06 (m, 6H), 6.65 (d, J=8.0 Hz, 2H), 3.99 (tdd, J=22.0, 8.6, 4.8 Hz, 4H), 3.65-3.53 (m, 5H), 3.48-3.24 (m, 19H), 3.03-2.83 (m, 7H), 2.42 (tt, J=11.9, 7.6 Hz, 2H), 2.31-2.02 (m, 16H), 1.98 (t, J=7.6 Hz, 5H), 1.86-1.63 (m, 8H), 1.62-1.40 (m, 1OH), 1.32 (m, 5H), 1.16 (m, 5H). ³¹P NMR (234 MHz, D₂O) δ 7.35. ¹³C NMR (151 MHz, D₂O) δ 183.1, 181.5, 181.4, 179.3, 178.5, 176.4, 175.0, 174.5, 173.8, 173.1, 172.7, 172.5, 150.5, 147.6, 141.5, 137.1, 132.2, 130.7, 129.2, 128.7, 128.5, 128.1, 126.9, 125.5, 122.5, 121.6, 114.7, 108.1, 91.4, 69.6, 69.5, 69.4, 66.7, 66.6, 64.4, 64.3, 56.5, 55.4, 54.9, 53.8, 39.3, 39.2, 38.7, 35.8, 35.7, 35.5, 35.4, 35.3, 34.3, 33.8, 33.7, 32.3, 32.1, 32.0, 31.9, 31.3, 28.2, 28.0, 27.8, 27.2, 27.0, 26.8, 26.7, 24.9, 24.8, 22.7. HRMS (MALDI): m/z calculated for C₇₂H₉₈IKN₉O₂₅P⁺[M+K]⁺: 1685.51729, found 1685.51514.

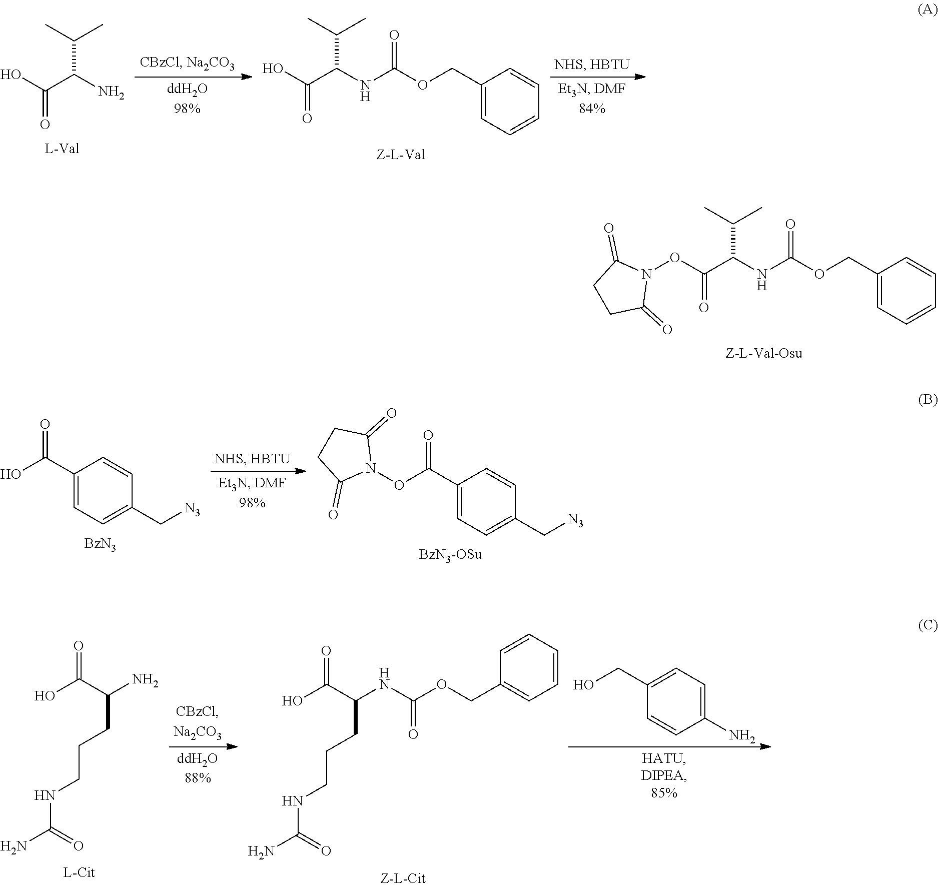

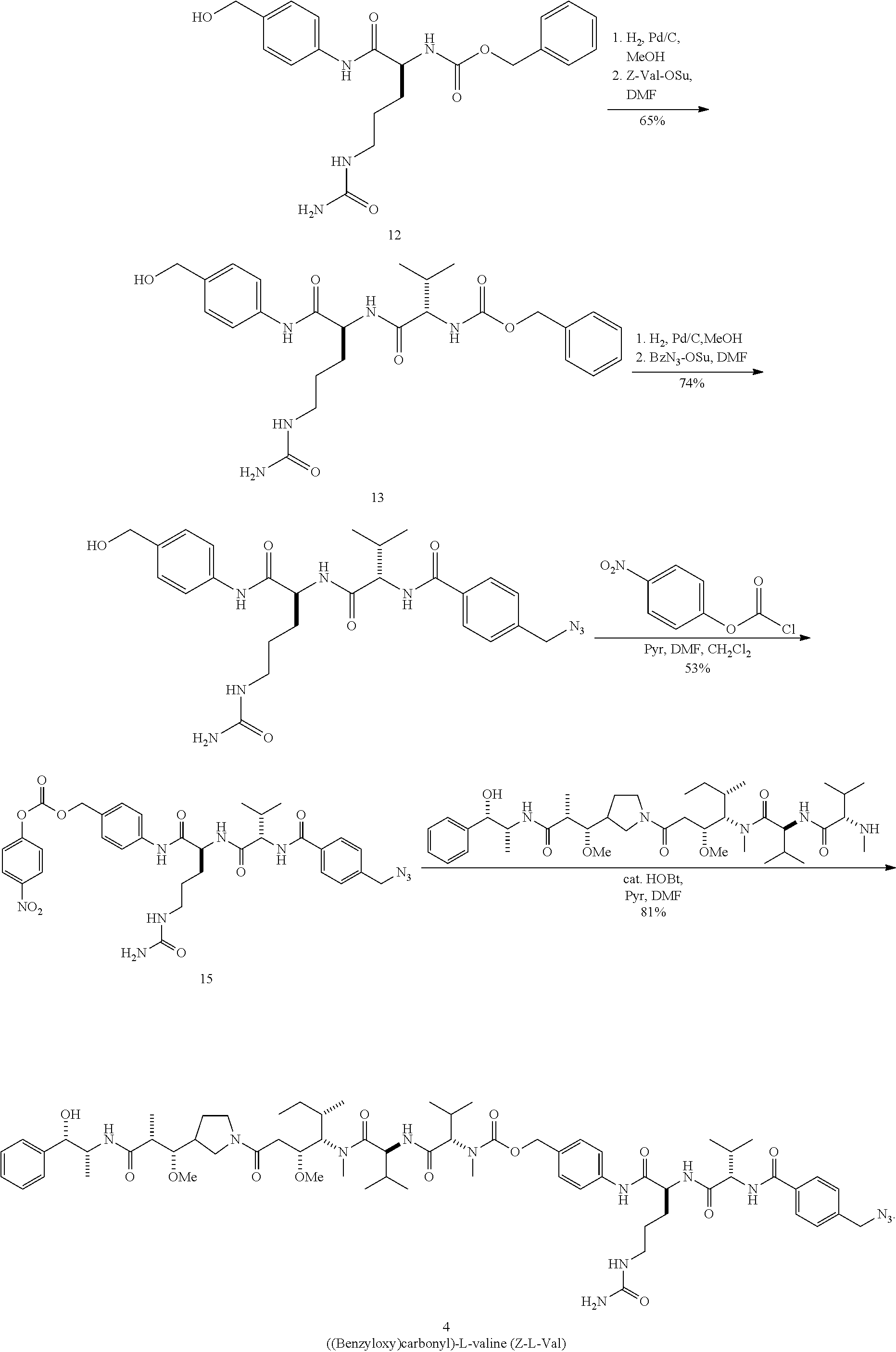

Following the procedure from Olatunji et al. (Tetrahedron Lett. 61, 152398 (2020)), L-Val (2 g, 17.1 mmol), (2.92 mL, 20.5 mmol), Na₂CO₃ (3.62 g, 34.1 mmol) and ddH₂O (30 mL) were used. The isolated yield was 98%. ¹H NMR (400 MHz, CDCl₃) δ 7.39-7.28 (m, 5H), 5.26 (d, J=9.1 Hz, 1H), 5.12 (s, 2H), 4.35 (dd, J=9.1, 4.5 Hz, 1H), 2.24 (dtd, J=13.8, 6.9, 4.1 Hz, 1H), 1.01 (d, J=6.9 Hz, 3H), 0.93 (d, J=6.9 Hz, 3H).

### 2,5-Dioxopyrrolidin-1-yl ((benzyloxy)carbonyl)-L-valinate (Z-L-Val-OSu)

A solution of Z-L-Val (2 g, 7.96 mmol) and HBTU (3.02 g, 7.96 mmol) was stirred in anhydrous DMF (14 mL) under Ar at room temperature for 30 min. A solution of N-hydroxyl succinimide (1.09 g, 9.55 mmol) and anhydrous Et₃N (2.08 mL, 11.9 mmol) in DMF (14 mL) was added to the reaction solution, and the reaction was stirred for 3 h under Ar. The reaction was diluted with EtOAc (180 mL) and washed sequentially with 1N HCl (75 mL, 3x), sat. NaHCO₃ (aq) (75 mL, 2×) and Brine (75 mL, 1×). Organic layer was dried on Na₂SO₄, volatiles were removed under reduced pressure, resulting residue was dried under high vacuum and carried on to the next step without further purification. Isolated yield was 84%. ¹H NMR (400 MHz, CDCl₃) δ 7.40-7.28 (m, 5H), 5.30-5.24 (m, 1H), 5.21-5.08 (m, 2H), 4.73-4.65 (m, 1H), 2.84 (s, 4H), 2.41-2.28 (m, 1H), 1.05 (2ds, J=6.9 Hz, 6H).

### 2,5-dioxopyrrolidin-1-yl 4-(azidomethyl)benzoate (BzN₃-Osu)

Following procedure for the preparation of Z-L-Val-NHS above, BzN₃ (0.6 g, 3.39 mmol), NHS (0.468 g, mmol), HBTU (1.28 g, 3.39 mmol), Et₃N (0.89 mL, 5.08 mmol) and DMF (22 mL) were used. Product was moved on to the next step without further purification. Isolated yield was 98%. ¹H NMR (400 MHz, CDCl₃) δ 8.20-8.13 (m, 2H), 7.47 (d, J=8.2 Hz, 2H), 4.47 (s, 2H), 2.92 (s, 4H).

### (S)-2-(((benzyloxy)carbonyl)amino)-5-ureidopentanoic acid (Z-L-Cit)

Following the procedure described in Olatunji et al., L-Cit (2 g, 17.1 mmol), CBzCl (1.96 mL, 13.7 mmol), Na₂CO₃ (2.42 g, 22.8 mmol) and ddH₂O (27 mL) were used. The isolated yield was 88%. ¹H NMR (400 MHz, DMSO-d₆) δ 12.58 (s, 1H), 7.61 (d, J=8.0 Hz, 1H), 7.42-7.26 (m, 5H), 5.93 (t, J=5.7 Hz, 1H), 5.38 (s, 2H), 5.03 (s, 2H), 3.93 (ddd, J=9.4, 7.9, 4.8 Hz, 1H), 3.19-3.14 (m, 1H), 2.93 (q, J=6.6 Hz, 2H), 1.75-1.62 (m, 1H), 1.61-1.48 (m, 1H), 1.40 (tdd, J=13.4, 8.5, 4.0 Hz, 2H).

### Benzyl (S)-(1-((4-(hydroxymethyl)phenyl)amino)-1-oxo-5-ureidopentan-2-yl)carbamate (12)

Following the procedure from Mondal et al. (Tetrahedron letters, 59(40), 3594-3599 (2018)), Z-L-Cit (3 g, 9.69 mmol), p-aminobenzyl alcohol (1.43 g, 11.6 mmol), HATU (4.43 g, 11.6 mmol), DIPEA (6.76 mL, 38.8 mmol) and DMF (18 mL) were used. The mixture was purified via silica-gel flash chromatography (10% MeOH, 30% CH₃CN in CH₂Cl₂→10% MeOH, 40% CH₃CN in CH₂Cl₂) to give a yellow solid in 85% yield. TLC: Rf=0.21 (10% MeOH, 40% CH₃CN in CH₂Cl₂, visualization by UV). ¹H NMR (400 MHz, DMSO-d₆) δ 9.98 (s, 1H), 7.56 (t, J=8.6 Hz, 3H), 7.41-7.14 (m, 7H), 5.98 (t, J=5.8 Hz, 1H), 5.42 (s, 2H), 5.10 (t, J=5.7 Hz, 1H), 5.03 (s, 2H), 4.43 (d, J=5.4 Hz, 2H), 4.14 (ddt, J=21.3, 10.5, 5.3 Hz, 1H), 3.17 (d, J=4.8 Hz, 1H), 2.98 (ddt, J=36.8, 13.2, 6.5 Hz, 2H), 1.59 (dtd, J=18.2, 14.4, 13.5, 8.6 Hz, 2H), 1.41 (2ds, J=6.9 Hz, 2H).

### Benzyl ((S)-1-(((S)-1-((4-(hydroxymethyl)phenyl)amino)-1-oxo-5-ureidopentan-2-yl)amino)-3-methyl-1-oxobutan-2-yl)carbamate (13)

Following the procedure from Mondal et al., 12 (2.65 g, 6.37 mmol), Pd/C (0.265 g, 10% mass eq.), Z-Val-Osu (2.14 g, 6.15 mmol), CH2Cl2 (40 mL), MeOH (160 mL) and DMF (22 mL) were used. The mixture was purified via silica-gel flash chromatography (10% MeOH, 10% CH₃CN in CH₂Cl₂→10% MeOH, 30% CH₃CN in CH₂Cl₂) to give a white solid in 65% yield. TLC: Rf=0.18 (10% MeOH, 40% CH₃CN in CH₂Cl₂, visualization by UV). ¹H NMR (400 MHz, DMSO-d6) δ 9.97 (s, 1H), 8.09 (d, J=7.6 Hz, 1H), 7.63-7.50 (m, 2H), 7.38-7.21 (m, 7H), 5.98 (t, J=5.8 Hz, 1H), 5.41 (s, 2H), 5.10 (t, J=5.7 Hz, 1H), 5.04 (s, 2H), 4.42 (t, J=6.0 Hz, 3H), 3.92 (dd, J=8.8, 6.8 Hz, 1H), 3.09-2.84 (m, 2H), 1.98 (h, J=6.7 Hz, 1H), 1.78-1.50 (m, 2H), 1.39 (ddt, J=22.1, 13.1, 8.3 Hz, 2H), 0.85 (2ds, J=6.8 Hz, 6H).

### 4-(azidomethyl)-N-((S)-1-(((S)-1-((4-(hydroxymethyl)phenyl)amino)-1-oxo-5-ureidopentan-2-yl)amino)-3-methyl-1-oxobutan-2-yl)benzamide (14)

Following the procedure from Mondal et al., 13 (1.47 g, 2.86 mmol), Pd/C (0.147 g, 10% mass eq.), BzN₃-Osu (0.94 g, 3.42 mmol), CH₂Cl₂ (70 mL), MeOH (700 mL) and DMF (10 mL). The mixture was purified via silica-gel flash chromatography (10% MeOH, 20% CH₃CN in CH₂Cl₂→10% MeOH, 40% CH₃CN in CH₂Cl₂) to give a white solid in 74% yield. TLC: Rf=0.20 (10% MeOH, 40% CH₃CN in CH₂Cl₂, visualization by UV). ¹H NMR (400 MHz, DMSO-d₆) δ 9.94 (s, 1H), 8.33 (d, J=8.4 Hz, 1H), 8.22 (d, J=7.6 Hz, 1H), 7.97-7.83 (m, 2H), 7.61-7.51 (m, 2H), 7.51-7.41 (m, 2H), 7.26-7.20 (m, 2H), 5.98 (t, J=5.8 Hz, 1H), 5.41 (s, 2H), 5.10 (t, J=5.7 Hz, 1H), 4.53 (s, 2H), 4.46-4.31 (m, 4H), 2.99 (ddq, J=25.8, 13.2, 6.6 Hz, 2H), 2.20-2.05 (m, 1H), 1.70 (dq, J=13.3, 7.9, 7.3 Hz, 1H), 1.60 (tq, J=9.1, 4.6 Hz, 1H), 1.53-1.28 (m, 2H), 0.94 (2ds, J=6.4 Hz, 6H). ¹³C NMR (101 MHz, DMSO-d₆) δ 171.2, 170.4, 166.3, 158.9, 138.9, 137.6, 137.4, 134.1, 128.2, 128.0, 130.0, 118.9, 62.6, 59.1, 53.2, 53.1, 30.2, 29.4, 26.8, 25.3, 19.4, 18.9. HRMS (MALDI): m/z calculated for C₂₆H₃₄N₈NaO₅⁺ [M+Na]⁺: 561.25444, found 561.25616; m/z calculated for C₂₆H₃₄KN₈O₅⁺ [M+K]⁺: 577.22837, found 577.22974.

### 4-((S)-2-((S)-2-(4-(azidomethyl)benzamido)-3-methylbutanamido)-5-ureidopentanamido)benzyl(4-nitro phenyl) carbonate (15)

To a stirring solution of 14 (0.2 g, 0.371 mmol) and pyridine (0.088 mL, 1.11 mmol) in anhydrous DMF (4 mL) under Ar was added a solution of p-nitrophenyl chloroformate (0.112 g, 0.557 mmol) in CH₂Cl₂ (1 mL) dropwise. The reaction was stirred overnight at ambient temperature. The solvent was removed under reduced pressure and the resulting mixture was purified via silica-gel flash chromatography (5% MeOH in CH₂Cl₂→8% MeOH in CH₂Cl₂) to give a white solid in 53% yield. TLC: Rf=0.35 (10% MeOH in CH₂Cl₂, visualization by UV). ¹H NMR (400 MHz, DMSO-d₆) δ 8.37-8.28 (m, 2H), 7.97-7.86 (m, 2H), 7.71-7.28 (m, 8H), 5.24 (s, 2H), 4.53 (s, 2H), 4.48-4.29 (m, 2H), 3.10-2.85 (m, 2H), 2.14 (hept, J=6.8 Hz, 1H), 1.78-1.56 (m, 2H), 1.56-1.32 (m, 2H), 0.95 (2ds, J=6.9, 6H). ¹³C NMR (101 MHz, DMSO-d₆) δ 171.2, 171.1, 170.8, 170.7, 170.6, 170.5, 166.3, 166.2, 158.9, 155.3, 151.9, 145.2, 139.4, 139.3, 138.9, 138.8, 134.1, 134.0, 132.4, 129.6, 129.3, 128.2, 128.0, 125.4, 122.7, 119.0, 118.9, 70.3, 59.0, 58.9, 53.1, 46.2, 30.2, 29.3, 26.8, 19.4, 18.9. HRMS (MALDI): m/z calculated for C₃₃H₃₇N₉NaO₉⁺ [M+Na]⁺: 726.26064, found 726.26434; m/z calculated for C₃₃H₃₇KN₉O₉[M+K]⁺: 742.23458, found 742.23602.

### 4-((S)-2-((S)-2-(4-(azidomethyl)benzamido)-3-methylbutanamido)-5-ureidopentanamido)benzyl((2S)-1-(((2S)-1-(((3R,4S,5S)-1-(3-((1S,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl) carbamate (4)

To a stirring solution of MMAE (0.122 g, 0.171 mmol), Pyridine (0.046 mL, 0.568 mmol) and HOBt hydrate (0.00435 g, 0.0284 mmol) in dry DMF (2 mL), was added a solution of 15 (0.1 g, 0.142 mmol) in DMF (2 mL) under at room temperature. Solvent was removed under reduced pressure and the crude product was purified via silica-gel flash chromatography (8% MeOH in CH₂Cl₂→10% MeOH in CH₂Cl₂) to give a colorless oil in 81% yield. TLC: Rf=0.21 (10% MeOH in CH₂Cl₂, visualization by UV). HRMS (MALDI): m/z calculated for C₆₆H₉₉N₁₃NaO₁₃⁺ [M+Na]⁺: 1304.73775, found 1304.74109; m/z calculated for C₆₆H₉₉KN₁₃O₁₃⁺ [M+K]⁺: 1320.71169, found 1320.70508.

### SMDC 1.

A solution of 4 (48 mg, 30.5 μmol) in (2 mL) was added a solution of 3 (50 mg, 32.5 μmol) in ddH₂O (2 mL); the reaction was stirred for 30 min at ambient temperature. Solvent was removed under reduced pressure and the resulting residue was purified by RP-Prep HPLC (5% CH₃CN in 10 mM NH₄OAc→100% CH₃CN, gradient shown below). Fractions containing the desired product were combined, the NH₄OAc was neutralized with excess KHCO₃ (approx. 2 eq), solvents were removed under reduced pressure and the resulting residue was lyophilized overnight. The solids were desalted using a C18 Sep-Pak (100% ddH₂O→100% MeOH) to yield an off-white solid product in 30%. ³¹P NMR (234 MHz, D₂O) δ 7.41. HRMS (MALDI): m/z calculated for C₁₃₈H₁₉₆IN₂₂O₃₈P²⁻/2 [M−2H]²⁻/2: 1463.6432, found 1463.6420; m/z calculated for C₁₃₈H₁₉₆IN₂₂O₃₈P³⁻/3 [M−3H]³⁻/3: 975.4262, found 975.4267; m/z calculated for C₁₃₈H₁₉₆IN₂₂O₃₈P⁴⁻/4 [M−4H]⁴⁻/4: 731.3177, found 731.3133.

### Example 2. Preparation and Characterization of SMDC 2

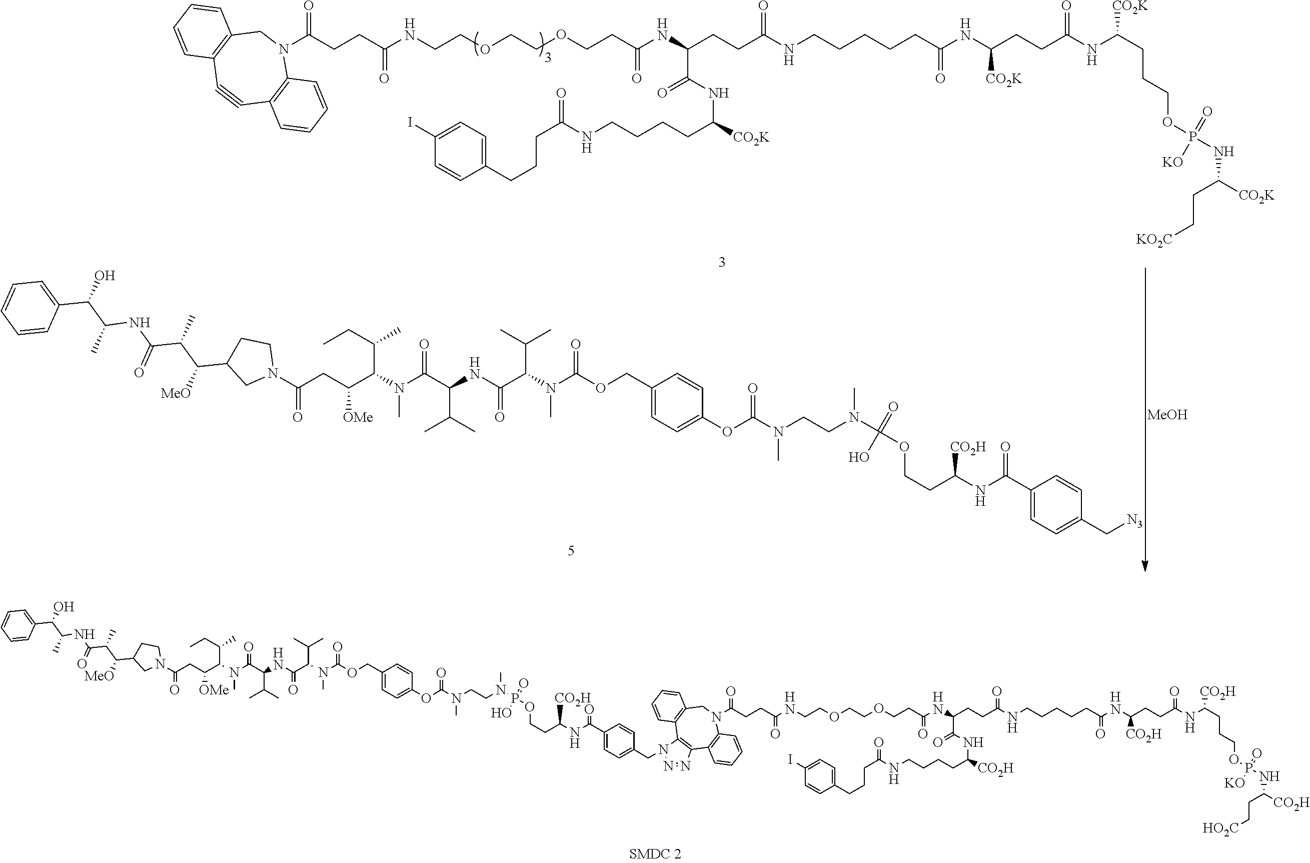

Following the synthesis and purification of 1 above, 3 (50 mg, 27.0 μmol), 5 (41 mg, 29.7 μmol) and ddH2O (4 mL) were used. Isolated yield was 49%. ³¹P NMR (234 MHz, D₂O) δ 9.59, 9.35, 7.42. HRMS (MALDI): m/z calculated for C₁₃₈H₁₉₃IN₂₀O₄₂P₂²⁻/2 [M−2H]²⁻/2: 1483.6051, found 1483.6007, found 1463.6420; m/z calculated for C₁₃₈H₁₉₃IN₂₀O₄₂P₂³⁻/3 [M−3H]³⁻/3: 988.7341, found 988.7234, found 975.4267; m/z calculated for C₁₃₈H₁₉₃IN₂₀O₄₂P₂⁴⁻/4 [M−4H]⁴⁻/4: 741.2986, found 741.2957.

### Example 3. PSMA Binding

Once characterized, IC₅₀ values for PSMA were determined as described previously and found to be in the nanomolar range for SMDC 1 (IC₅₀=24.1±2.1 nM) and SMDC 2 (IC₅₀=1.74±0.14 nM), which were consistent with other PSMA inhibitors of this class.

### Example 4. Evaluation of SMDC 1 and SMDC 2 in Preclinical Models of Human PCa

Efficacy Study Design: NCr nude mice (age 6-8 weeks) injected with 1×10⁶ PC3 PiP cells (1:1 in saline with Matrigel, 100 μL total volume) subcutaneously to right rear flank. Tumors were allowed to grow until approximate size of 100 mm³ (roughly 2-3 weeks) before starting compound treatments. Treatment consisted of an injection of the appropriate compound to the lateral tail vein (100 μL bolus) once a week for the first 6 weeks of the study. Efficacy treatment groups were 8 animals each of: 1) saline, 2) MMAE alone, 0.2 mg/kg (molar equivalent to the MMAE conjugated to the small molecules), 3) CTT1700 (structure noted below), 0.8 mg/kg, 4) 1, 0.8 mg/kg, and 5) 2, 0.8 mg/kg. Tumor sizes were measured twice weekly for the duration of the study and mortality noted for survival curves. Animals were sacrificed if tumor condition reached IACUC standards for euthanasia (tumor burden of >1.5 cm in any axis, ulceration, >20% weight loss). Blood draws were taken via superficial temporal vein at 0, 4, 7 and 10 weeks and CBCs with 3-part differential measured.

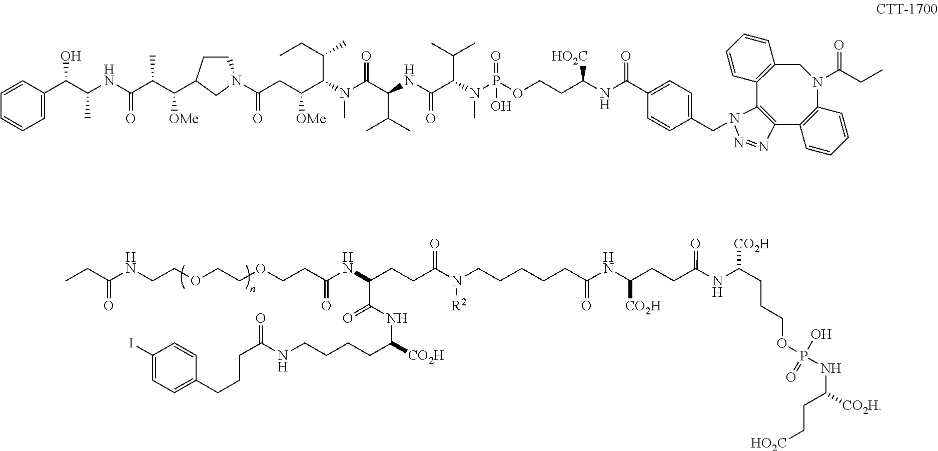

Compound Residence study design (PK Studies): Three volumes of PBS, pH 7.4, were added to tumor, kidney, and lacrimal tissues and 10 volumes of PBS, pH 7.4, were added to prostate tissue samples. Tumor, kidney, and prostate samples were homogenized with a homogenizer, and lacrimal tissues were sonicated with a sonicator. Twenty five microliters of plasma or tissue homogenate was added to a microcentrifuge tube followed by 10 μL of internal standard solution and 100 μL of 5 mM ammonium bicarbonate and acetonitrile (25:75 v,v). The sample was then vortexed at a high setting for 1 minute and centrifuged at 17,200×g for 10 minutes. The resultant supernatant was pipetted into an HPLC vial and 10-20 μL was injected into the LC-MS/MS system. SMDC 2 was used as internal standard for the quantitation of CTT1700 and SMDC 1, and SMDC 1 was used as internal standard for SMDC 2. [²H₈]-MMAE was used as the internal standard for MMAE. CTT1700 (MW 3013.4 g/mol), SMDC 2 (MW 3275.7 g/mol) and SMDC 1 (MW 3159.6 g/mol) concentrations were quantitated using a SCIEX (Framingham, MA 01701) 4500 LC-MS/MS system consisting of an Exion HPLC and 4500 mass detector. A SCIEX 6500+ Qtrap LC-MS/MS system consisting of an Agilent (Palo Alto, CA 94036) 1290 HPLC and Sciex 6500+ QTrap mass detector was used for quantitation of MMAE concentrations. The chromatography consisted of a gradient using of 5 mM ammonium bicarbonate and acetonitrile and the separation was achieved using a Phenomenex (Torrance, CA 90501) Kinetex column (2×50 mm, 2.6 μm). The MRM m/z transitions monitored were 903.1/419.0, 990.7/419.0, 977.8/718.4, 718.5/686.5 and 726.5/694.5 for CTT1700, SMDC 2, SMDC 1, MMAE and [²H₃]-MMAE, respectively. Standard curves were linear from 10-10,000 ng/mL for CTT1700, SMDCs 2 and 1 while the MMAE standard curves were linear from 0.01 to 10 ng/mL. A 1/y² weighted linear regression was used for all standard curves.

PK parameters were estimated using standard noncompartmental methods with Phoenix WinNonlin (Certara, Princeton, NJ). Plasma parent drugs were derived using the IV bolus approach, while all other data were derived using the extravascular approach.

### DISCUSSION

To evaluate efficacy of SMDCs 1 and 2 in preclinical models of human PCa, we employed xenograft tumor models derived from PSMA+ PC3-PIP tumor cells implanted subcutaneously in the right rear flank of NCr nude mice (6-8 weeks old). Three weeks after injection of the cells, the average tumor volume reached 100 mm³, and mice were treated with 0.8 mg/kg of SMDCs (252.8 nmol/kg of SMDC 1, 244.2 nmol/kg of SMDC 2, n=8) via lateral tail vein injection (100 μL bolus) once weekly for six weeks. Tumor sizes were measured twice weekly for the duration of the study and mortality noted for survival curves for 90 days after first administration. Animals were euthanized if tumor or mouse condition reached IACUC standards for euthanasia (tumor burden of >1.5 cm in any axis, ulceration, >20% mouse weight loss). Blood draws were taken via superficial temporal vein at 0, 4, 7 and 10 weeks, and complete blood count (CBC) analysis was performed with a 3-part differential measurement. For biodistribution studies, blood samples were obtained at 1 and 6 h post a single 0.8 mg/kg dose, followed by euthanasia at 24, 72 and 120 h; LC-MS/MS analysis was performed to determine concentrations of SMDC and MMAE in plasma, tumor, lacrimal gland, kidney, and prostate samples.

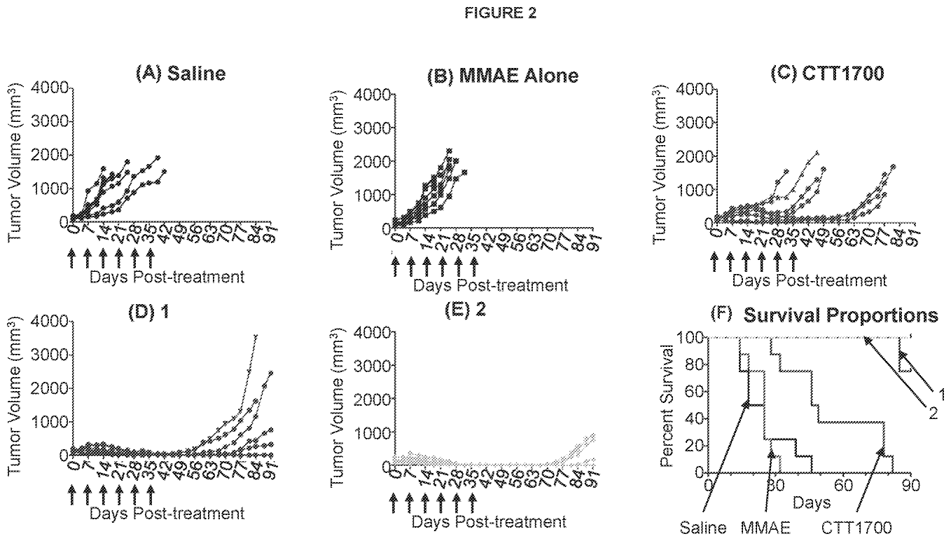

Tumor growth and survival data for mice administered with controls and SMDCs 1 and 2 are shown in FIG. 2. Animals in the saline and MMAE control groups reached their end point criteria within 6 weeks of their first administration (FIGS. 2A and 2B). This was to be expected for MMAE administration, since the cytotoxic agent is known to be non-specific, with minimal accumulation of this agent expected in the tumor and microenvironment. Animals that were treated with CTT1700 (a PSMA-targeted SMDC with a stable phosphoramidate linker) showed moderate efficacy, with all animals reaching end point tumor burden within 12 weeks of dosing initiation (FIG. 2C). In sharp contrast, SMDCs 1 and 2 showed dramatic efficacy compared to controls. For animals treated with compound 1, tumor regression was observed following initiation of administration up until 2 weeks after the last dose, with subsequent slow tumor growth. Six of eight mice survived 90 days after initial dosing (FIG. 2D). Animals treated with compound 2 showed significant tumor growth regression, with no detectable tumor in any of the mice by day 35 of first dosage (FIG. 2E). The survival of mice in SMDC 2 group were statistically greater than the other groups, with all animals surviving at 90 days (FIG. 2F). It is important to note that in all groups of mice, their weights remained constant or increased during the survival period, there were no overt visual signs of toxicity (scruffy coat, diarrhea, lethargy), and CBCs were in the normal range (data not shown). Based on these data and observations, the dose regimen uniformly utilized for this comparative study is below the maximum tolerated dose (MTD) in immune-compromised nude mice.

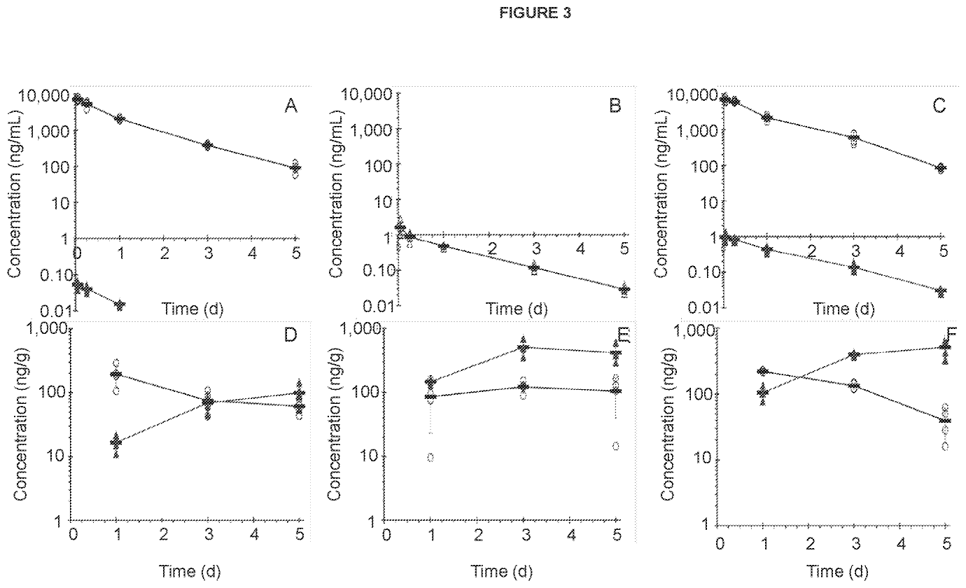

Plasma and tissue biodistribution are shown in FIG. 3. The plasma levels of SMDCs and released MMAE give an indication of overall exposure, while levels in the kidneys and lacrimal glands represent organs where off-target effects with PSMA-targeted agents has been observed in the past. Parent SMDCs 1 and 2 exhibited a distribution volume of 0.106 and 0.122 L/kg, respectively, a value in between plasma volume and extracellular water volume. This is indicative of the albumin binding motif enhancing half-life and retention of the SMDCs in plasma. Generally, observed plasma MMAE and parent drug profiles exhibited parallel and roughly mono-exponential profiles. Furthermore, we observed drug-released MMAE elimination half-lives (t_{1/2}) from plasma of 13 h and 24 h for SMDC 1 and 2 respectively, much higher than the t_{1/2} of 4.1 h reported for MMAE when dosed directly. These observations are indicative of a formation rate limited elimination, with the pharmacokinetics (PK) of the parent drug determining and prolonging MMAE exposure. Plasma concentrations of the MMAE were found to be 5 orders of magnitude lower than the parent drugs indicating low systemic release of MMAE payload. In tumor, concentration of the parent drugs were roughly in the same order of magnitude suggesting similar efficiency in targeting, while MMAE concentrations varied between compounds according to linker stability. A slow decline of CTT1700 and 2 in all tissues over the course of 5 days was observed. Surprisingly, SMDC 1 could not be quantified in any other tissue other than the tumor (Table 1). MMAE concentrations in tissues appeared to remain constant or decline over time, while MMAE concentrations in tumor continue to show steady increase between days 1 and 5, for all parent drugs. This is indicative of the slow turnover and selective accumulation of MMAE from the parent SMDCs in the tumor tissue preferentially to the other tissues. Moreover, SMDCs 1 and 2 resulted in approximately 6-fold higher absolute tumor MMAE exposure compared the control SMDC with a non-degradable linker, CTT1700. The reported plasma clearance of MMAE of 60 mL/h corresponds to 47 L/d/kg, allows us to use the apparent MMAE formation clearance we observed in our data, in order to calculate the fraction of parent compound that is converted to MMAE. The fraction of drug that is estimated to release MMAE for CTT1700, SMDCs 1 and 2 was 1%, 38%, and 36%, respectively. This 40-fold difference in MMAE-releasing ability between CTT1700 relative to P and 2 was reflected in MMAE tumor concentrations, arguably, the most important metric for interpreting the efficacy data.

TABLE 1
Plasma and tumor pharmacokinetic parameters of parent drug and MMAE payload after
IV dosing of 0.8 mg/kg CTT-1700, VC, or PH to NCr nude mice with PC3-PiP tumors.

| | CTT1700 | | SMDC 1 | | SMDC 2 | |
|---|---|---|---|---|---|---|
| | Parent | MMAE | Parent | MMAE | Parent | MMAE |
| Plasma | | | | | | |
| Cₘₐₓ (ng/mL) | 7025 | 0.053 | <10 | 1.60 | 7078 | 0.95 |
| Tₘₐₓ (h) | 1 | 1 | | 1 | 1 | 1 |
| AUC₀₋ₜ (ng/mL · d) | 6442 | 0.0300 | — | 1.42 | 7434 | 1.31 |
| AUC_{0-inf} (ng/mL · d) | 6566 | 0.0417 | — | 1.46 | 7520 | 1.35 |
| t_{1/2} (h) | 23.0 | 12.9 | — | 23.8 | 17.0 | 22.4 |
| Cl (L/d/kg) | 0.122 | 4569* | — | 125* | 0.106 | 130* |
| Vss (L/kg) | 0.136 | — | — | — | 0.121 | — |
| % extrapolated | 1.9 | 28 | — | 2.9 | 1.1 | 3.1 |
| Tumor | | | | | | |
| Cₘₐₓ (ng/g) | 194 | 98.7 | 122 | 509 | 222 | 518 |
| AUC₀₋ₜ (ng/g · d) | 483 | 262 | 475 | 1652 | 613 | 1471 |
| Kₚ (tumor/plasma) | 0.0750 | 8733 | — | 1163 | 0.083 | 1123 |
| Prostate | | | | | | |
| Cₘₐₓ (ng/g) | 280 | 0.914 | — | 8.14 | 399 | 7.54 |
| AUC₀₋ₜ (ng/g · d) | 647 | 2.99 | — | 20.8 | 869 | 22.7 |
| Kₚ (prostate/plasma) | 0.100 | 99.7 | — | 14.6 | 0.117 | 17.3 |
| Kidney | | | | | | |
| Cₘₐₓ (ng/g) | 400 | 1.82 | — | 30.6 | 1066 | 26.5 |
| AUC₀₋ₜ (ng/g · d) | 685 | 4.00 | — | 55.5 | 2140 | 62.2 |
| Kₚ (kidney/plasma) | 0.106 | 133 | — | 39.1 | 0.288 | 47.5 |
| Lacrimal | | | | | | |
| Cₘₐₓ (ng/g) | 71.9 | 0.340 | — | 1.59 | 353 | 5.50 |
| AUC₀₋ₜ (ng/g · d) | 220 | 1.13 | — | 5.39 | 620 | 12.7 |
| Kₚ (Lacrimal/plasma) | 0.0342 | 37.7 | — | 3.80 | 0.0834 | 9.69 |

VC concentrations in all tissues other than tumor were below the LLOQ.
*CL/F, where F represents the fraction of drug that resulted in release of MMAE; value corrected for difference in molecular weight.

In summary, the pharmacokinetics performance of the pH cleavable linker in 2 can be correlated with its superior efficacy when compared to the control CTT1700. The data indicate that efficient release of the payload from 2 reflects an optimal design of its acid-labile linker technology. In addition, the in vivo performance of SMDC 2 compared to that of SMDC 1 demonstrates that the acid-labile phosphoramidate linker is competitive with the Val-Cit linker that is prevalent in ADCs. Though the efficacy and biodistribution experiments conducted are proof-of-concept studies, the results obtained warrant exploration of this novel acid-labile linker technology in the context of releasing other clinically-relevant payloads, as well as further rigorous pharmacokinetics studies prior to advancement into the clinic.

### Examples 5-29. Additional SMDCs According to the Disclosure

Additional examples are prepared essentially according to the procedures noted above, where an azide is reacted with a targeting molecule to result in the corresponding triazole-containing SMDC. Two targeting molecules, or their salts, are as follows:

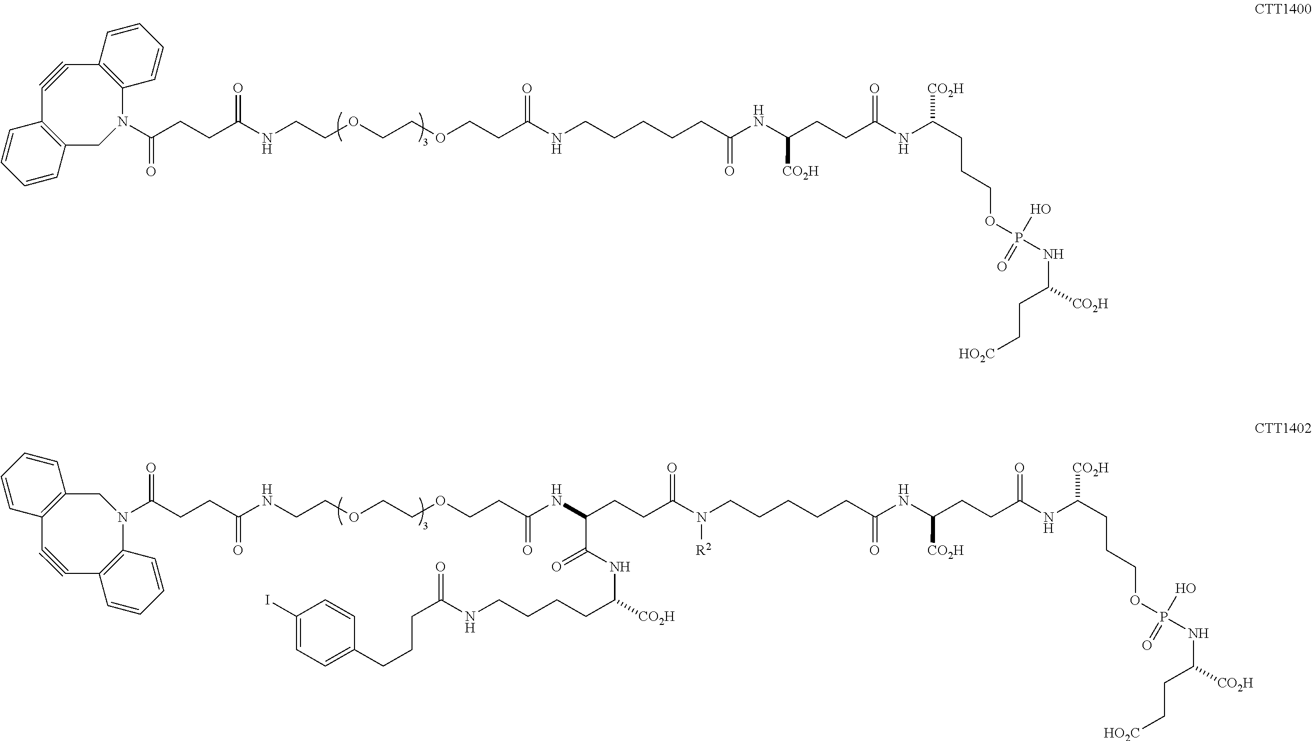

| Ex. | | Targeting |
|---|---|---|
| No. | Azide | molecule |
| 5 | Compound 4 | CTT1400 |
| 6 | Compound 5 (MMAE-PhosAm-A) | CTT1400 |
| 7 | 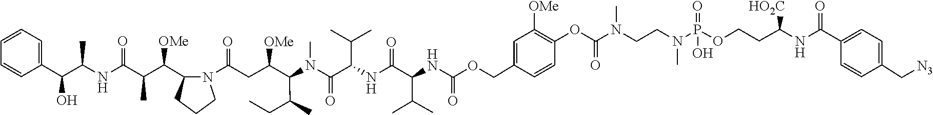 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC(=O)OCc1ccc(OC(=O)N(C)CCN(C)P(=O)(O)OCC[C@H](NC(=O)c2ccc(CN=[N+]=[N-])cc2)C(=O)O)c(OC)c1)C(C)C)C(C)C] | CTT1400 |
| 8 | Doxorubicin-PhosAm-A: | CTT1400 |
| | 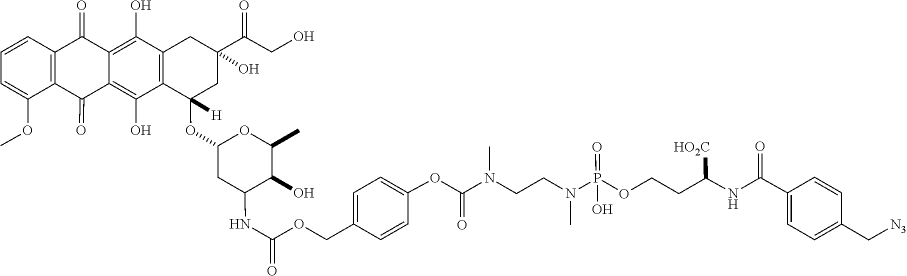 [SMILES: COc1cccc2c1C(=O)c1c(O)c3c(c(O)c1C2=O)C[C@@](O)(C(=O)CO)C[C@@H]3O[C@H]1CC(NC(=O)OCc2ccc(OC(=O)N(C)CCN(C)P(=O)(O)OCC[C@H](NC(=O)c3ccc(CN=[N+]=[N-])cc3)C(=O)O)cc2)[C@H](O)[C@H](C)O1] | |
| 9 | (R)-9b-PhosAm-A: | CTT1400 |
| | 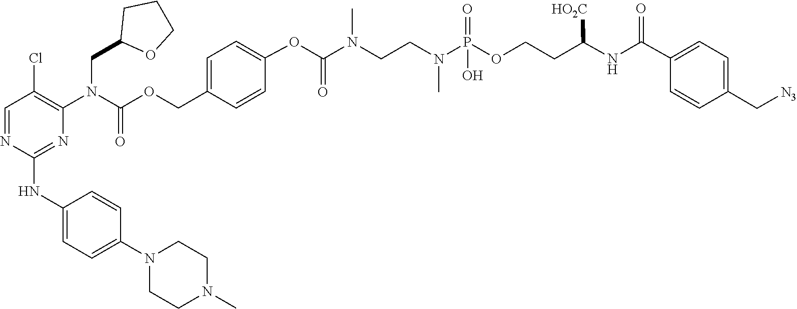 | |
| 10 | MMAF-PhosAm-A: | CTT1400 |
| | 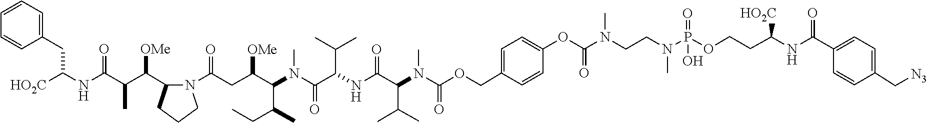 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OCc1ccc(OC(=O)N(C)CCN(C)P(=O)(O)OCC[C@H](NC(=O)c2ccc(CN=[N+]=[N-])cc2)C(=O)O)cc1)C(C)C] | |
| 11 | Cabazitaxel-PhosAm-B: | CTT1400 |
| | 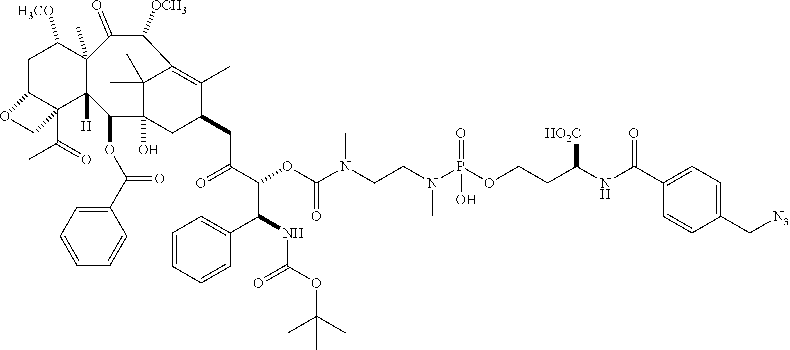 | |
| 12 | Docetaxel-PhosAm-B: | CTT1400 |
| | 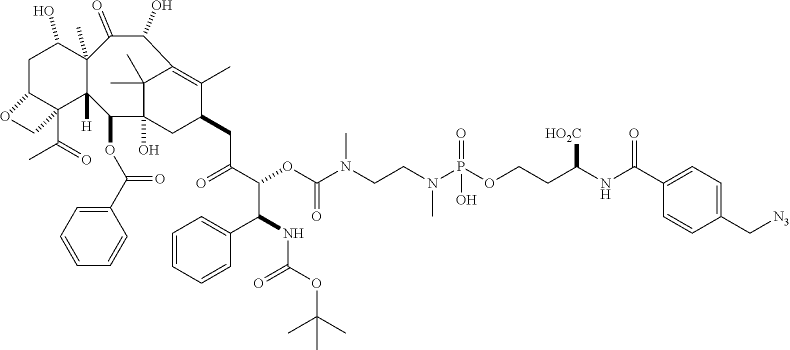 | |
| 13 | Paclitaxel-PhosAm-B: | CTT1400 |
| | 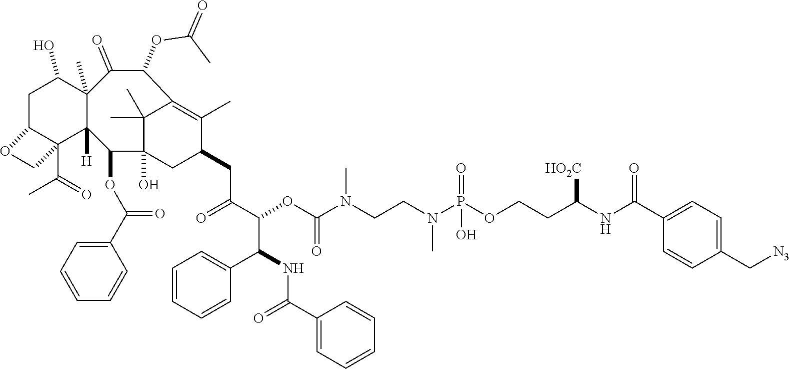 | |
| 14 | SN-38-PhosAm-B: | CTT1400 |
| | 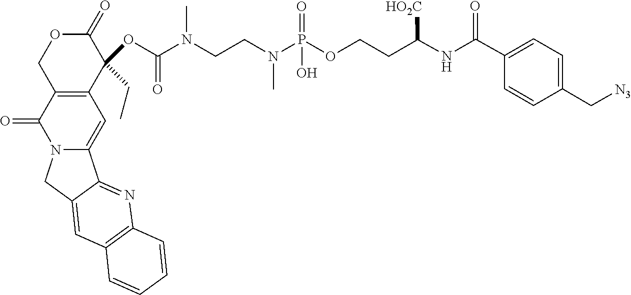 [SMILES: CC[C@@]1(OC(=O)N(C)CCN(C)P(=O)(O)OCC[C@H](NC(=O)c2ccc(CN=[N+]=[N-])cc2)C(=O)O)C(=O)OCc2c1cc1n(c2=O)Cc2cc3ccccc3nc2-1] | |
| 15 | Gemcitabine-PhosAm-B: | CTT1400 |
| | 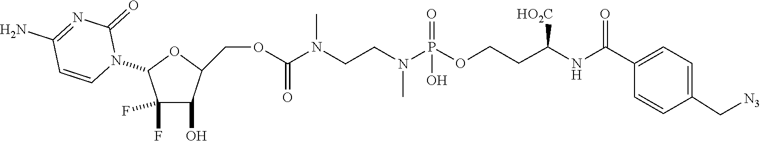 | |
| 16 | seco-DUBA-PhosAm-B: | CTT1400 |
| | 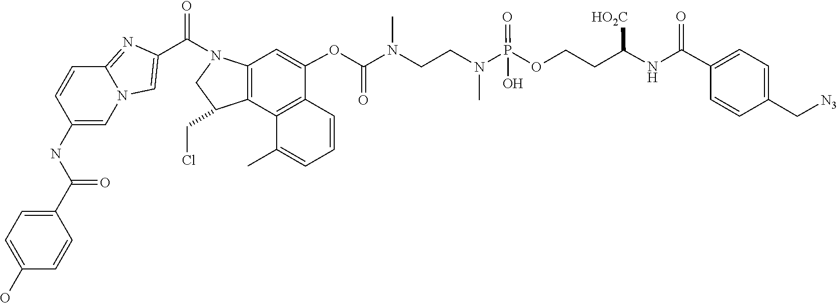 [SMILES: Cc1cccc2c(OC(=O)N(C)CCN(C)P(=O)(O)OCC[C@H](NC(=O)c3ccc(CN=[N+]=[N-])cc3)C(=O)O)cc3c(c12)[C@H](CCl)CN3C(=O)c1cn2cc(NC(=O)c3ccc(O)cc3)ccc2n1] | |
| 17 | Imiquimod-PhosAm-A: | CTT1400 |
| | 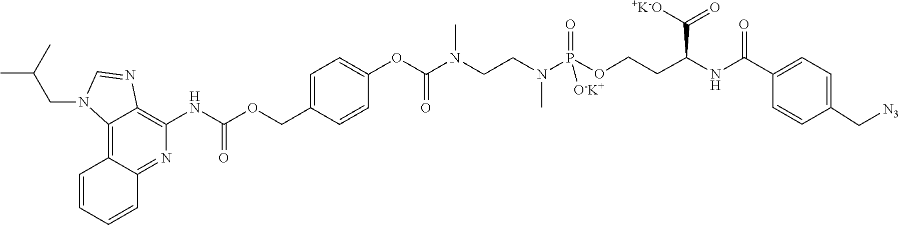 | |
| 18 | Doxorubicin-PhosAm-A | CTT1402 |
| 19 | (R)-9b-PhosAm-A | CTT1402 |
| 20 | MMAF-PhosAm-A | CTT1402 |
| 21 | Cabazitaxel-PhosAm-B | CTT1402 |
| 22 | Docetaxel-PhosAm-B | CTT1402 |
| 23 | Paclitaxel-PhosAm-B | CTT1402 |
| 24 | SN-38-PhosAm-B | CTT1402 |
| 25 | Gemcitabine-PhosAm-B | CTT1402 |
| 26 | seco-DUBA-PhosAm-B | CTT1402 |
| 27 | Imiquimod-PhosAm-A | CTT1402 |
| 28 | 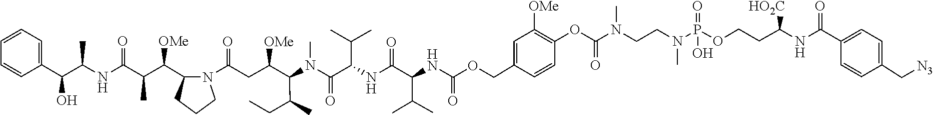 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC(=O)OCc1ccc(OC(=O)N(C)CCN(C)P(=O)(O)OCC[C@H](NC(=O)c2ccc(CN=[N+]=[N-])cc2)C(=O)O)c(OC)c1)C(C)C)C(C)C] | CTT1402 |
| 29 | 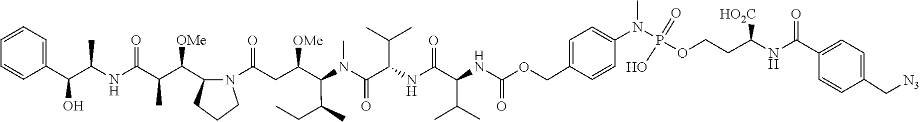 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC(=O)OCc1ccc(N(C)P(=O)(O)OCC[C@H](NC(=O)c2ccc(CN=[N+]=[N-])cc2)C(=O)O)cc1)C(C)C)C(C)C] | CTT1402 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of structural Formula (I) 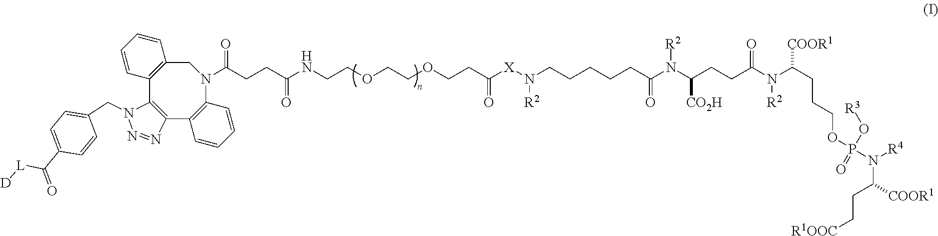
or a pharmaceutically acceptable salt thereof, wherein
D is a therapeutic or diagnostic agent attached to L through —NR²—, —S—, or —O— moiety;
L is an acid-cleavable linker;
X is a bond or 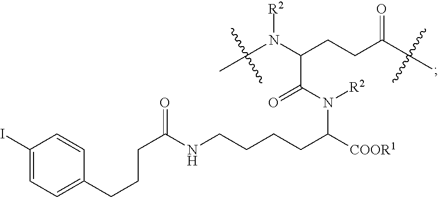
n is in a range of 1 to 6; and
R¹, R², R³, and R⁴ are independently H, -C₁-C₆ alkyl, or a protecting group.

2. (canceled)

3. The compound according to claim 1, wherein D is an anti-angiogenic agent, a cytotoxic agent, a cytokine, a chemokine, an apoptotic agent, a prodrug, a toxin, an enzyme, a radioisotope, an immunomodulator, an antibiotic, an agent active in the CNS or a hormone or D is an imaging agent, a fluorescent dye, a near-IR dye, an enzyme, a chemiluminescent agent, a bioluminescent agent, a paramagnetic ion, an ultrasound label, or a radioacoustic label.

4. The compound according to claim 1, wherein D is monomethylauristatin E (MMAE), monomethylauristatin F (MMAF), doxorubicin, cabazitaxel, docetaxel, paclitaxel, gemcitabine, imiquimod, 7-ethyl-10-hydroxycamptothecin (SN-38), duocarmycin (DUBA), seco-duocarmycin (seco-DUBA), (R)-5-Chloro-N²-[4-(4-methylpiperazin-1-yl)phenyl]-N⁴-[(tetrahydrofuran-2-yl)methyl]pyrimidine-2,4-diamine ((R)-9b), or gemcitabine.

5. (canceled)

6. (canceled)

7. The compound according to claim 1, wherein D is attached to L through —NR²—.

8. The compound according to claim 1, wherein L is selected from: 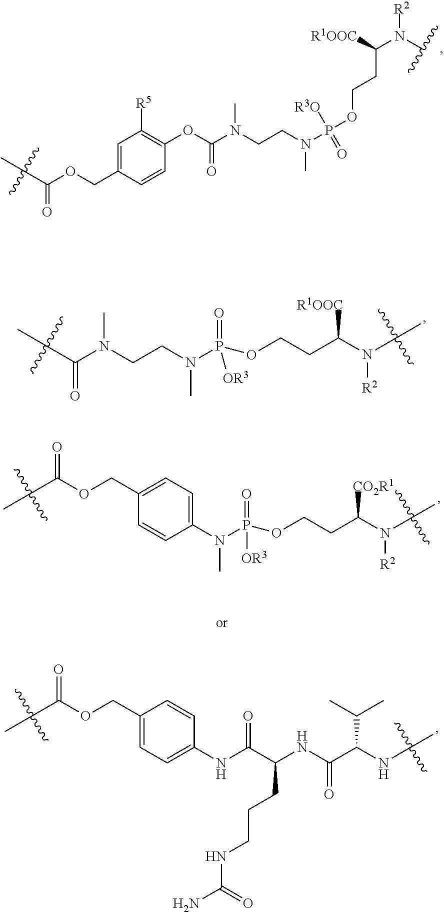
where R⁵ is H, —OH, or —OC₁-C₆ alkyl.

9. (canceled)

10. The compound according to claim 1, wherein L is 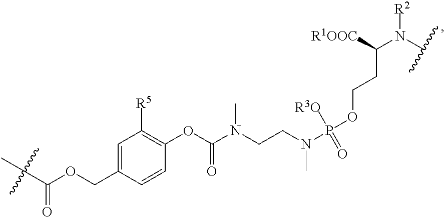
where R⁵ is H, —OH, or —OC₁-C₆ alkyl.

11. The compound according to claim 1, wherein L is

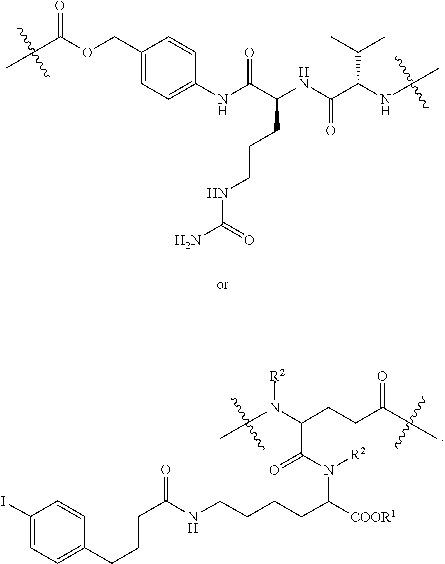

12. (canceled)

13. The compound of claim 11, which is of formula: 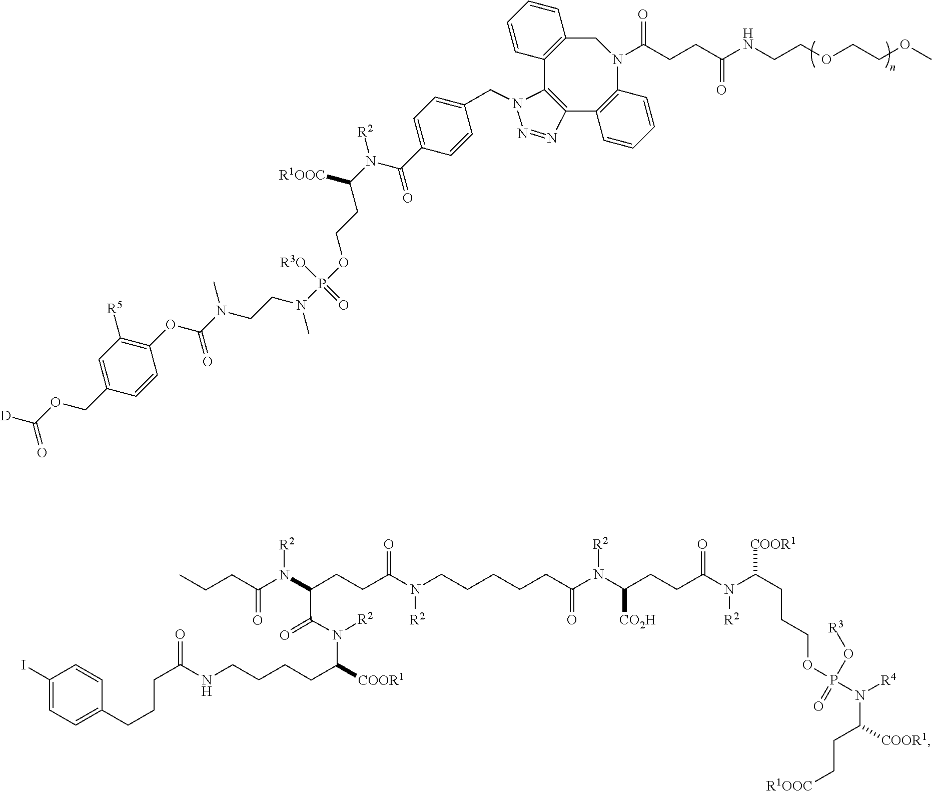
where R⁵ is H, —OH, or —OC₁-C₆ alkyl.

14. The compound of claim 8, wherein R⁵ is H or —OCH₃.

15. (canceled)

16. The compound according to claim 1, wherein R¹, R², and R³ are independently H or —C₁-C₄ alkyl.

17. (canceled)

18. (canceled)

19. The compound according to claim 1, wherein R⁴ is H or methyl.

20. (canceled)

21. The compound according to claim 1, of formula:

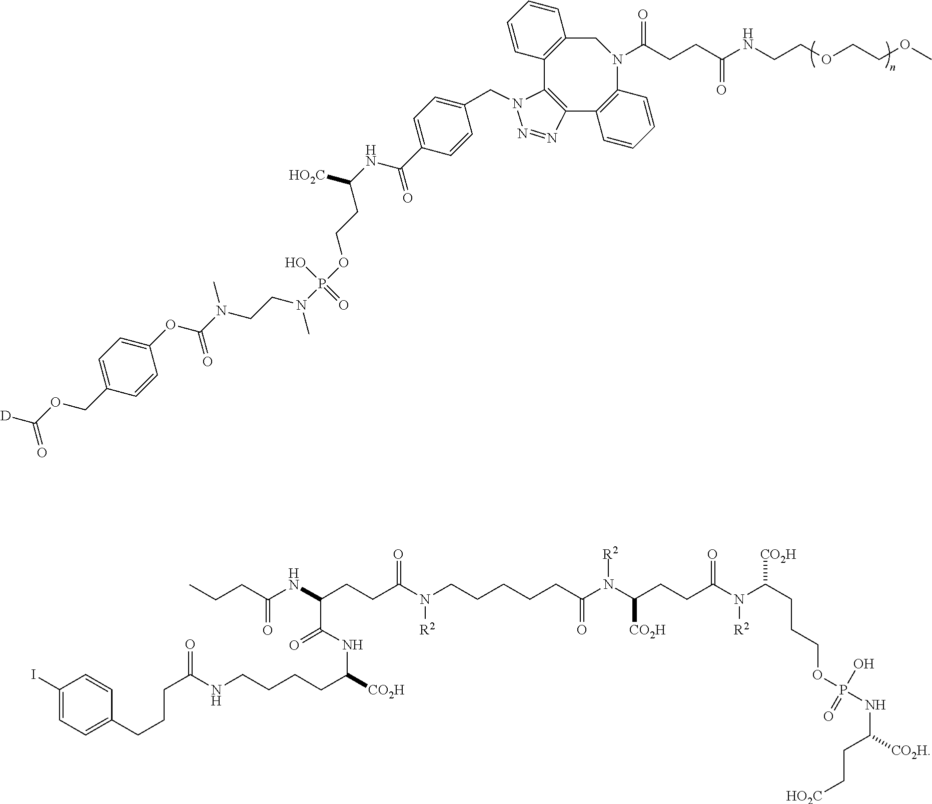

22. The compound according to claim 1, of formula:

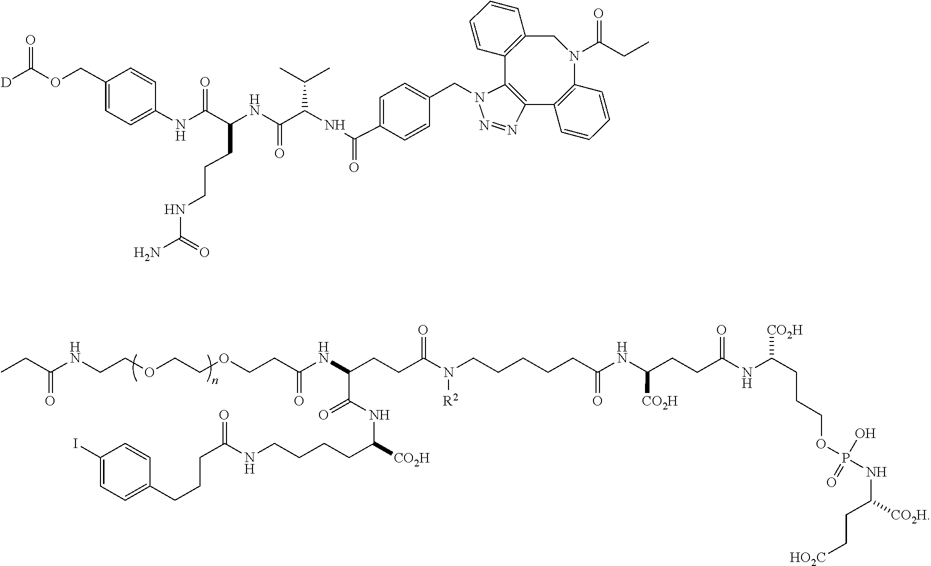

23. The compound of claim 21, wherein D is MMAE.

24. (canceled)

25. (canceled)

26. A pharmaceutical composition comprising a compound of claim 1 and a pharmaceutically acceptable excipient, carrier, adjuvant, stabilizer, and/or diluent.

27. A method of delivering a therapeutic or diagnostic agent to a subject, wherein the method comprises administering a therapeutically effective amount of a compound according to claim 1 or a pharmaceutical composition comprising a compound of claim 1 and a pharmaceutically acceptable excipient, carrier, adjuvant, stabilizer, and/or diluent.

28. A method of treating a patient with prostate cancer, the method comprising administering to the patient an effective amount of a compound according to claim 1 or a pharmaceutical composition comprising a compound of claim 1 and a pharmaceutically acceptable excipient, carrier, adjuvant, stabilizer, and/or diluent.

29. A method for imaging one or more prostate cancer cells in a patient comprising administering to the patient a compound according to claim 1 or a pharmaceutical composition comprising a compound of claim 1 and a pharmaceutically acceptable excipient, carrier, adjuvant, stabilizer, and/or diluent.

30. A method a synthesizing a compound of formula (I) as described in claim 1, the method comprising contacting a compound of formula (II) 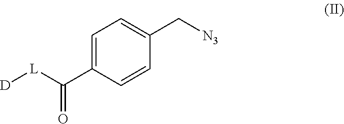
or a pharmaceutically acceptable salt thereof,
with a compound of formula (III) 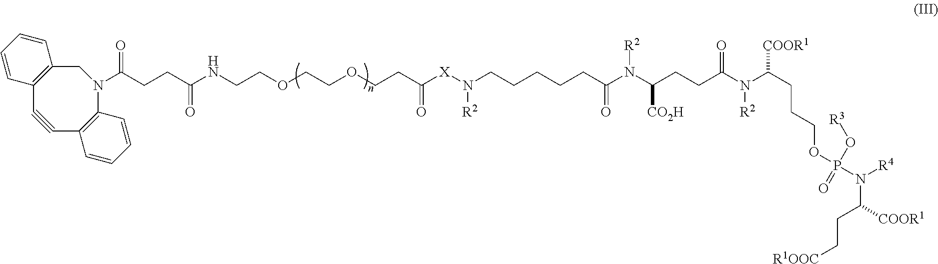
or a pharmaceutically acceptable salt thereof, wherein D, L, n, R¹, R², R³,
and R⁴ are as provided above with respect to formula (I).

31. (canceled)

32. The compound of claim 22, wherein D is MMAE.
